(19) United States
(12) Patent Application Publication
(10) Pub. No.: US 2026/0047947 A1
(43) Pub. Date: Feb. 19, 2026

(54) DEVICE FOR COMPRESSING AND LOADING A STENT

(71) Applicant: BOSTON SCIENTIFIC SCIMED, INC., Maple Grove, MN (US)

(72) Inventors: Levi Joel Wolterstorff, Saint Paul, MN (US); Eric Michael Petersen, Maple Grove, MN (US); Joseph Edward Adriaens, Elk River, MN (US)

(73) Assignee: BOSTON SCIENTIFIC SCIMED, INC., MAPLE GROVE, MN (US)

(21) Appl. No.: 19/299,935

(22) Filed: Aug. 14, 2025

Related U.S. Application Data
(60) Provisional application No. 63/683,394, filed on Aug. 15, 2024.

(51) Int. Cl. A61F 2/95

(52) U.S. Cl. CPC A61F 2/9524

(57) ABSTRACT

A device for radially compressing a stent includes a housing and a first iris positioned adjacent the housing. The first iris includes a circumferential ring, a plurality of arms extending radially inward from the ring, and a lever extending radially outward from the ring. The first iris includes an open position recess, a closed position recess, and a guide recess formed in the ring. The device may include a second iris positioned adjacent the housing, and a spacer plate disposed between the first and second irises. The spacer plate includes first and second locking elements disposed radially inward of the outer circumference of the spacer plate. The first locking element engages with the first iris to selectively lock in open and closed positions. The second locking element engages with the second iris to selectively lock in open and closed positions. The first and second locking elements are circumferentially spaced apart.

### CROSS-REFERENCE TO RELATED APPLICATIONS

This application claims the benefit of priority of U.S. Provisional Application No. 63/683,394 filed Aug. 15, 2024, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a device and/or method for radially compressing and/or loading a stent and/or a stent device such as a replacement heart valve implant.

### BACKGROUND

Conventional stent crimping devices have been used throughout the medical device industry to crimp balloon expandable stents, self-expanding stents, replacement heart valve implants, etc. Conventional stent crimping devices have intricate arrangements of multiple parts and/or difficult assemblies and rely upon complex interactions between many moving parts. The cost of a conventional stent crimping device may be significant due to the aforementioned complexity and number of components required and/or used in a conventional stent crimping device. Of the known devices and methods for compressing and/or crimping stents, each has certain advantages and disadvantages. There is an ongoing need for alternative devices and/or methods for compressing stents, stent devices, and/or other medical implants that may include a stent, such as but not limited to replacement heart valve implants.

### SUMMARY

In a first example, a device for radially compressing a stent may comprise a housing including a central opening, and a first iris positioned adjacent the housing. The first iris may include a first circumferential ring positioned coaxially relative to the central opening, a first plurality of arms extending radially inward from the first circumferential ring, and a first lever extending radially outward from the first circumferential ring. The first iris may include a first open position recess formed in the first circumferential ring, a first closed position recess formed in the first circumferential ring, and a first guide recess formed in the first circumferential ring.

In addition, or alternatively, to any example described herein, the first guide recess is disposed circumferentially between the first open position recess and the first closed position recess.

In addition, or alternatively, to any example described herein, the first guide recess includes a first open position ramp, a first closed position ramp, and a first central recess disposed between the first open position ramp and the first closed position ramp.

In addition, or alternatively, to any example described herein, a first locking element is configured to selectively engage with the first open position recess, the first closed position recess, and the first guide recess.

In addition, or alternatively, to any example described herein, the first locking element includes a first deflectable tab extending radially relative to the central opening and a first rounded projection extending from the first deflectable tab, wherein the first deflectable tab is disposed radially inward of an outer circumference of the first circumferential ring.

In addition, or alternatively, to any example described herein, the first rounded projection is configured to extend into the first open position recess when the first iris is disposed in a first open position.

In addition, or alternatively, to any example described herein, more force is required to move the first rounded projection from the first open position recess to the first guide recess than from the first guide recess to the first open position recess.

In addition, or alternatively, to any example described herein, the first rounded projection is configured to extend into the first closed position recess when the first iris is disposed in a first closed position.

In addition, or alternatively, to any example described herein, more force is required to move the first rounded projection from the first closed position recess to the first guide recess than from the first guide recess to the first closed position recess.

In addition, or alternatively, to any example described herein, the first open position recess, the first closed position recess, and the first guide recess extend into the first circumferential ring in a direction parallel to a central longitudinal axis of the housing.

In addition, or alternatively, to any example described herein, the first open position recess, the first closed position recess, and the first guide recess are disposed radially inward of an outer circumference of the first circumferential ring.

In addition, or alternatively, to any example described herein, the first open position recess, the first closed position recess, and the first guide recess are disposed within an opposing half of the first iris from the first lever.

In addition, or alternatively, to any example described herein, and in a second example, a device for radially compressing a stent may comprise a housing including a central opening, a first iris positioned adjacent the housing, a second iris positioned adjacent the housing, and a spacer plate disposed between the first iris and the second iris. The spacer plate may include a first locking element disposed radially inward of an outer circumference of the spacer plate and a second locking element disposed radially inward of the outer circumference of the spacer plate. The first locking element may be engaged with the first iris to selectively lock the first iris in a first open position and a first closed position. The second locking element may be engaged with the second iris to selectively lock the second iris in a second open position and a second closed position.

In addition, or alternatively, to any example described herein, the first iris includes a first circumferential ring positioned coaxially relative to the central opening, a first plurality of arms extending radially inward from the first circumferential ring, and a first lever extending radially outward from the first circumferential ring.

In addition, or alternatively, to any example described herein, the second iris includes a second circumferential ring positioned coaxially relative to the central opening, a second plurality of arms extending radially inward from the second circumferential ring, and a second lever extending radially outward from the second circumferential ring.

In addition, or alternatively, to any example described herein, the first locking element includes a first deflectable tab extending radially relative to the central opening and a first rounded projection extending from the first deflectable tab in a first direction parallel to a central longitudinal axis of the housing.

In addition, or alternatively, to any example described herein, the second locking element includes a second deflectable tab extending radially relative to the central opening and a second rounded projection extending from the second deflectable tab in a second direction parallel to the central longitudinal axis of the housing.

In addition, or alternatively, to any example described herein, the second direction is opposite the first direction.

In addition, or alternatively, to any example described herein, more force is required to unlock the first iris from the first open position than to lock the first iris in the first open position, and more force is required to unlock the first iris from the first closed position than to lock the first iris in the first closed position.

In addition, or alternatively, to any example described herein, more force is required to unlock the second iris from the second open position than to lock the second iris in the second open position, and more force is required to unlock the second iris from the second closed position than to lock the second iris in the second closed position.

In addition, or alternatively, to any example described herein, the first locking element and the second locking element are monolithically formed with the spacer plate.

In addition, or alternatively, to any example described herein, and in a third example, a device for radially compressing a stent may comprise a housing including a central opening, a first iris positioned adjacent the housing, wherein the first iris includes a first circumferential ring positioned coaxially relative to the central opening, a first plurality of arms extending radially inward from the first circumferential ring, and a first lever extending radially outward from the first circumferential ring, a second iris positioned adjacent the housing, wherein the second iris includes a second circumferential ring positioned coaxially relative to the central opening, a second plurality of arms extending radially inward from the second circumferential ring, and a second lever extending radially outward from the second circumferential ring, and a spacer plate disposed between the first iris and the second iris. The spacer plate may include a first locking element disposed radially inward of an outer circumference of the spacer plate and a second locking element disposed radially inward of the outer circumference of the spacer plate, the second locking element being circumferentially spaced apart from the first locking element. The first locking element may be engaged with the first circumferential ring to selectively lock the first iris in a first open position and a first closed position. The second locking element may be engaged with the second circumferential ring to selectively lock the second iris in a second open position and a second closed position.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a device for radially compressing a stent;

FIG. 2 is an exploded view illustrating selected aspects of the device of FIG. 1;

FIGS. 3-5 illustrate selected aspects of the device of FIGS. 1-2;

FIGS. 6-7 illustrate selected aspects related to the function of the device of FIGS. 1-5;

FIGS. 8A-8C are detailed partial cut away views illustrating selected aspects related to the function of the device of FIGS. 1-5; and

FIGS. 9-13 are schematic partial cross-sectional views illustrating selected aspects of a method of radially compressing a stent using the device of FIGS. 1-2.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the arm”, “the aperture”, or other features may be equally referred to all instances and quantities beyond one of said feature unless clearly stated to the contrary. As such, it will be understood that the following discussion may apply equally to any and/or all of the components for which there are more than one within the device, etc. unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below. For the purpose of this disclosure, the discussion below is directed toward a device and method for radially compressing a stent and will be so described in the interest of brevity. This, however, is not intended to be limiting as the skilled person will recognize that the following discussion may also apply to stent devices or medical implants including a stent with no or minimal changes to the structure and/or scope of the disclosure. Similarly, the devices and methods disclosed herein may have applications and uses for other medical devices.

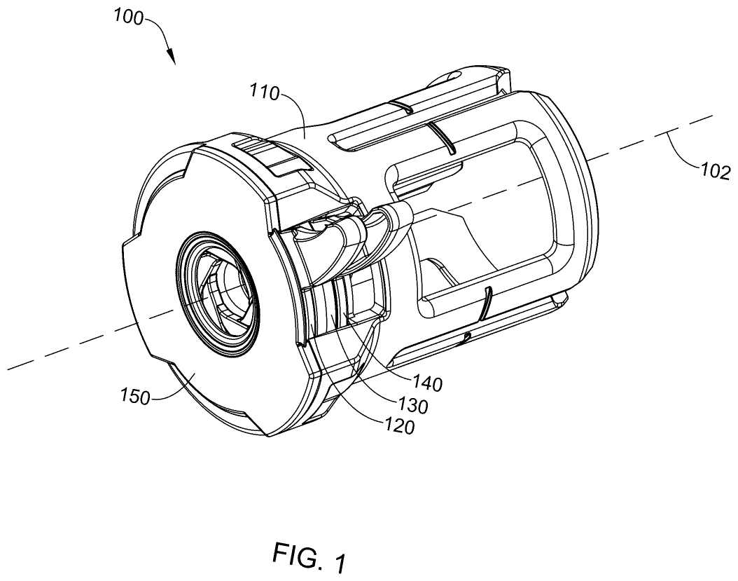

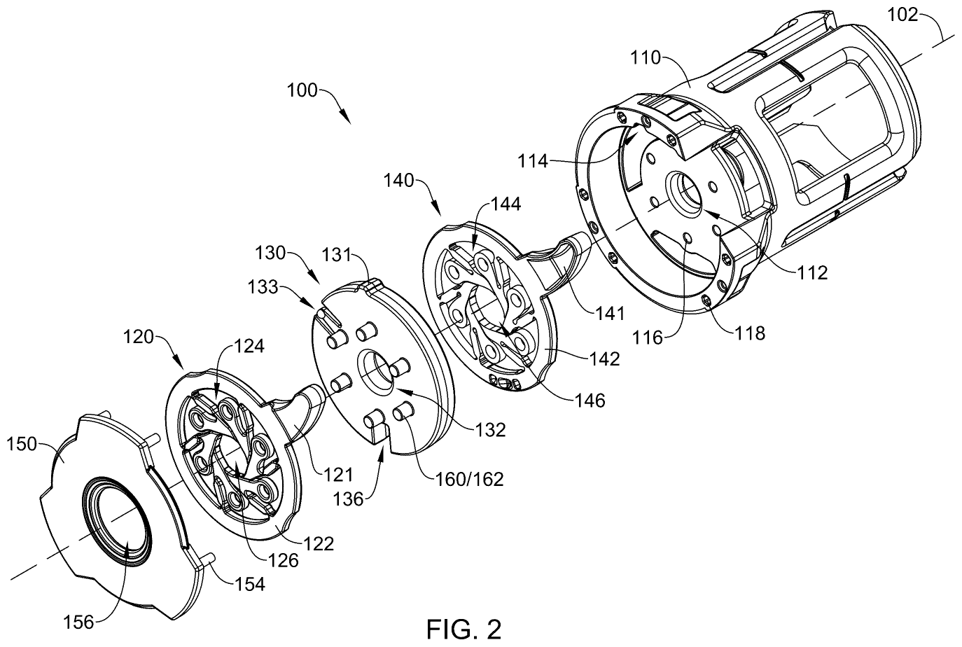

FIGS. 1-5 illustrate aspects of a device 100 for radially compressing a stent. FIG. 2 is an exploded view of the device 100 shown in FIG. 1. It should be noted that some elements of the device 100 have been omitted from illustration to improve clarity, and some elements may be shown in and/or may only be visible in some figures and not in others. Some reference numbers may be shown in some figures only for reference purposes and the elements referred thereto may be described with respect to other figures.

The device 100 may comprise a housing 110 including a central opening 112 aligned with and/or disposed coaxially with a central longitudinal axis 102 of the device 100 and/or the housing 110. In some embodiments, the device 100 may comprise a first iris 120 positioned adjacent the housing 110. In at least some embodiments, the first iris 120 may be positioned at least partially within the housing 110. In some embodiments, the device 100 may comprise a spacer plate 130 positioned adjacent the housing 110. In at least some embodiments, the spacer plate 130 may be positioned at least partially within the housing 110. In some embodiments, the device 100 may comprise a second iris 140 positioned adjacent the housing 110. In at least some embodiments, the second iris 140 may be positioned at least partially within the housing 110. In some embodiments, the device 100 may comprise a cover plate 150 positioned adjacent the housing 110. In some embodiments, the cover plate 150 may be positioned at least partially within the housing 110.

In some embodiments, the cover plate 150 may include a cover plate opening 156, as seen in FIG. 2. The cover plate opening 156 may be aligned with and/or may be positioned coaxially relative to the central opening 112 of the housing 110. In some embodiments, the cover plate 150 may be non-rotatable relative to the housing 110. In some embodiments, the cover plate 150 may be removably secured to the housing 110. In some embodiments, the cover plate 150 may be removably secured to the housing 110 using one or more fasteners (not shown). In some embodiments, the cover plate 150 may include a plurality of openings (not shown) extending along a perimeter of the cover plate 150. The plurality of openings may be configured to receive fasteners therein to removably secure and/or removably attach the cover plate 150 to the housing 110. The housing 110 may include a plurality of corresponding holes or recesses aligned with the plurality of openings in the cover plate 150 and configured to receive the fasteners therein. In one example, the fasteners may be externally threaded screws or other threaded fasteners and the plurality of corresponding holes or recesses may include internal threads configured to threadably engage the externally threaded screws or other threaded fasteners. Other configurations and/or fastener types are also contemplated. In some embodiments, the cover plate 150 may include one or more protrusions 154 extending from the cover plate 150 that may be configured to engage one or more apertures 118, slots, or other features formed in the housing 110 to removably secure the cover plate 150 to the housing 110.

In some embodiments, the one or more protrusions 154 may be configured to form an interference fit and/or a friction fit with the one or more apertures 118, slots, or other features formed in the housing 110 to removably secure the cover plate 150 to the housing 110. Other configurations are also contemplated. Some suitable but non-limiting examples of materials that may be used to for the housing 110 and/or the cover plate 150, including but not limited to metals and metal alloys, composites, ceramics, polymers, and the like, are described below.

In some embodiments, the first iris 120 may be movable with respect to the housing 110, the spacer plate 130, and/or the cover plate 150. In some embodiments, at least a portion of the first iris 120 may be configured to rotate relative to the housing 110, the spacer plate 130, and/or the cover plate 150. In some embodiments, the second iris 140 may be movable with respect to the housing 110, the spacer plate 130, and/or the cover plate 150. In some embodiments, at least a portion of the second iris 140 may be configured to rotate relative to the housing 110, the spacer plate 130, and/or the cover plate 150. In some embodiments, at least a portion of the second iris 140 may be configured to rotate relative to the first iris 120. Accordingly, in at least some embodiments, the first iris 120 and the second iris 140 may be movable independently of each other. In some embodiments, the first iris 120 may be movable and/or may be configured to rotate relative to the housing 110, the spacer plate 130, and/or the cover plate 150 independently of the second iris 140. In some embodiments, the second iris 140 may be movable and/or may be configured to rotate relative to the housing 110, the spacer plate 130, and/or the cover plate 150 independently of the first iris 120.

In some embodiments, the first iris 120 and the second iris 140 may be movable and/or may be configured to rotate relative to the housing 110, the spacer plate 130, and/or the cover plate 150 together, in tandem, and/or simultaneously. In some embodiments, the first iris 120 and the second iris 140 may be movable and/or may be configured to rotate relative to the housing 110, the spacer plate 130, and/or the cover plate 150 together, in tandem, and/or simultaneously at one time, in one circumferential direction, or during a particular step of a method disclosed herein, and the first iris 120 and the second iris 140 may be movable and/or may be configured to rotate relative to the housing 110, the spacer plate 130, and/or the cover plate 150 independently of each other at another time, in another direction, or during another particular step of a method disclosed herein. Other configurations are also contemplated.

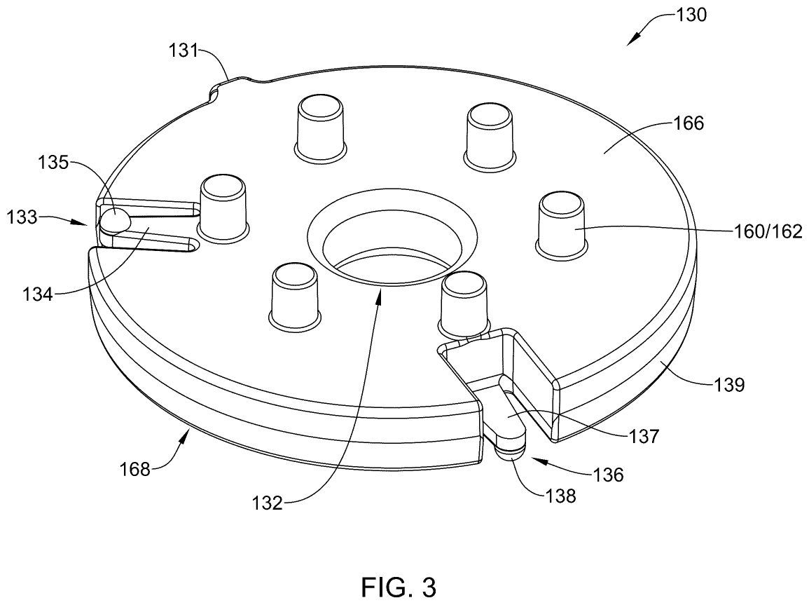

FIG. 3 illustrates selected aspects of the spacer plate 130 in more detail. In some embodiments, the spacer plate 130 may be non-rotatable relative to the housing 110. In some embodiments, the spacer plate 130 may engage the housing 110 as discussed herein to prevent relative rotation therebetween. In some embodiments, the second iris 140 may be axially offset from the first iris 120. In some embodiments, the spacer plate 130 may be disposed between the first iris 120 and the second iris 140. In some embodiments, the second iris 140 may be axially offset from the first iris 120. In some embodiments, the second iris 140 may be spaced apart from the first iris 120. In some embodiments, the second iris 140 may be spaced apart from the first iris 120 by the spacer plate 130. Other configurations are also contemplated.

In some embodiments, the spacer plate 130 may include a central opening 132 formed therein. In some embodiments, the central opening 132 of the spacer plate 130 may be aligned with and/or may be positioned coaxially relative to the central opening 112 of the housing 110, a first central opening 126 of the first iris 120, and/or the central longitudinal axis 102, as seen in FIG. 2. In some embodiments, the spacer plate 130 may be disposed axially between the first iris 120 and the housing 110 with respect to the central longitudinal axis 102. In some embodiments, the spacer plate 130 may be configured to slide axially into the housing 110. The housing 110 may include a notch or a recess 114 (e.g., FIG. 2) formed in a sidewall of the housing 110. The spacer plate 130 may include a locking tab 131 projecting radially outward from an outer perimeter and/or an outer circumference 139 of the spacer plate 130. In some embodiments, the outer perimeter and/or the outer circumference 139 of the spacer plate 130 may be substantially circular. The locking tab 131 may be configured to engage with the recess 114 of the housing 110 to prevent relative rotation between the spacer plate 130 and the housing 110.

In some embodiments, the device 100 and/or the spacer plate 130 may include a plurality of projections 160. In some embodiments, the plurality of projections 160 may extend axially from the spacer plate 130 (e.g., parallel to the central longitudinal axis 102). In some embodiments, the plurality of projections 160 may be removable from and/or configured to be disengaged from the spacer plate 130. In at least some embodiments, the plurality of projections 160 may be fixedly attached to the spacer plate 130. In some embodiments, the plurality of projections 160 may be integrally and/or monolithically formed with the spacer plate 130. In some embodiments, the plurality of projections 160 may comprise a first plurality of projections 162 extending in a first direction parallel to the central longitudinal axis 102 of the device 100 and/or the housing 110 from the spacer plate 130 and/or an upper surface 166 of the spacer plate 130 (e.g., extending upward from the spacer plate 130 and/or the upper surface 166 of the spacer plate 130). In some embodiments, the plurality of projections 160 may comprise a second plurality of projections (not visible) extending in a second direction parallel to the central longitudinal axis 102 of the device 100 and/or the housing 110 opposite the first direction from the spacer plate 130 and/or a lower surface 168 of the spacer plate 130 (e.g., extending downward from the spacer plate 130 and/or the lower surface 168 of the spacer plate 130. In some embodiments, the plurality of projections 160 may comprise the first plurality of projections 162 and the second plurality of projections. In some embodiments, the first plurality of projections 162 and the second plurality of projections may collectively comprise and/or constitute the plurality of projections 160. The plurality of projections 160 may be configured to engage the housing 110, the first iris 120, the second iris 140, and/or the cover plate 150. In some embodiments, the first plurality of projections 162 may be configured to engage the first iris 120 and/or the cover plate 150. In some embodiments, the second plurality of projections may be configured to engage the second iris 140 and/or the housing 110. In at least some embodiments, the housing 110 may include a plurality of apertures 116 (e.g., FIG. 2) configured to receive the second plurality of projections extending distally from the spacer plate 130. In some embodiments, the plurality of apertures 116 may be arranged in an array around the central opening 112. Other configurations are also contemplated.

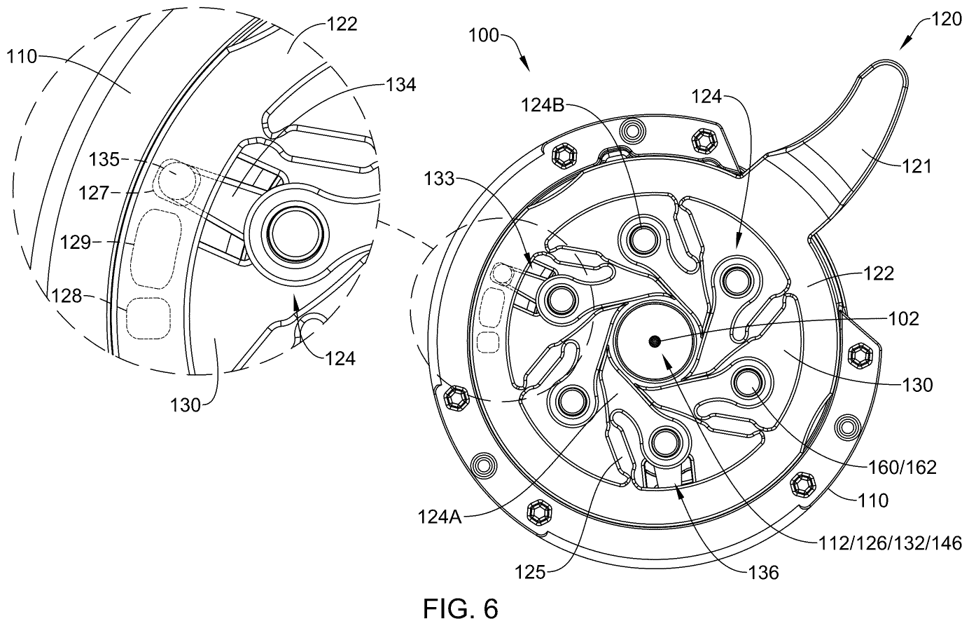

In some embodiments, the device 100 and/or the spacer plate 130 may comprise a first locking element 133 configured to engage and/or be engaged with the first iris 120. In some embodiments, the first locking element 133 may be disposed radially inward of the outer perimeter and/or the outer circumference 139 of the spacer plate 130. In some embodiments, the first locking element 133 may include a first deflectable tab 134 extending radially relative to the central opening 112 of the housing 110 and/or the central opening 132 of the spacer plate 130, and a first rounded projection 135 extending from the first deflectable tab 134. In some embodiments, the first rounded projection 135 may extend perpendicularly from the first deflectable tab 134. In some embodiments, the first rounded projection 135 may extend from the first deflectable tab 134 in the first direction parallel to the central longitudinal axis 102 of the device 100 and/or the housing 110. In some embodiments, the first rounded projection 135 may be hemispherical in shape. Other configurations are also contemplated. In at least some embodiments, the first rounded projection 135 may extend to an uppermost extent above and/or upward of the upper surface 166 of the spacer plate 130. In some embodiments, the first locking element 133 may be monolithically formed with the spacer plate 130. Additional details regarding the first locking element 133 and/or its function are described herein with respect to FIG. 6.

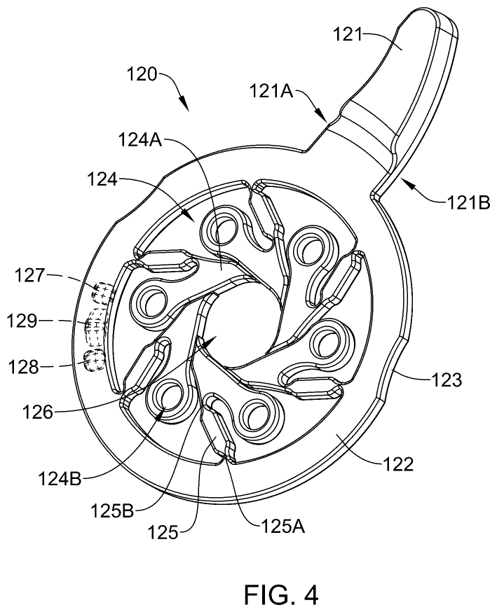

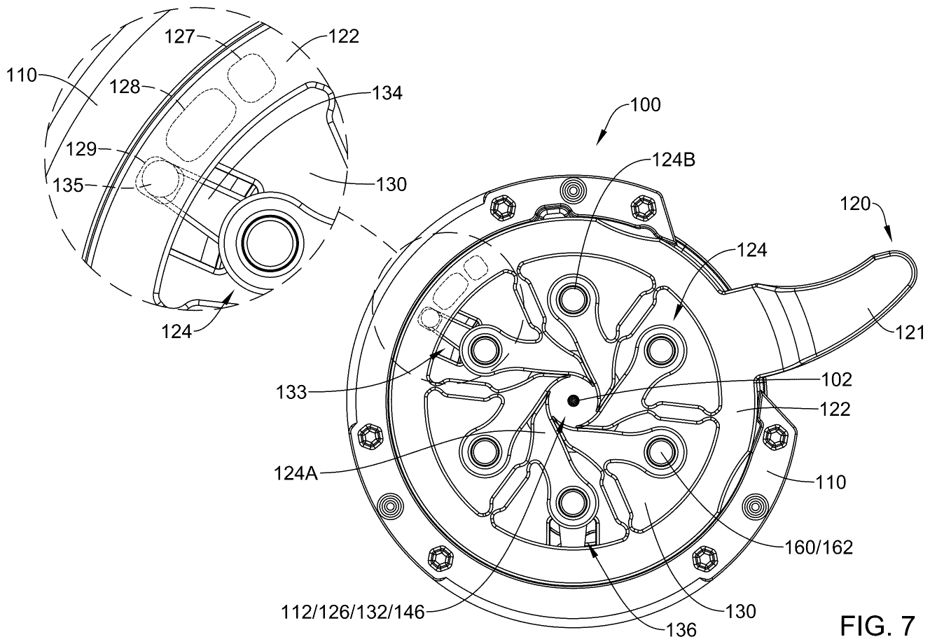

In some embodiments, the device 100 and/or the spacer plate 130 may comprise a second locking element 136 configured to engage and/or engaged with the second iris 140. In some embodiments, the second locking element 136 may be disposed radially inward of the outer perimeter and/or the outer circumference 139 of the spacer plate 130. In some embodiments, the second locking element 136 may include a second deflectable tab 137 extending radially relative to the central opening 112 of the housing 110 and/or central opening 132 of the spacer plate 130, and a second rounded projection 138 extending from the second deflectable tab 137. In some embodiments, the second rounded projection 138 may extend perpendicularly from the second deflectable tab 137. In some embodiments, the second rounded projection 138 may extend from the second deflectable tab 137 in the second direction parallel to the central longitudinal axis 102 of the device 100 and/or the housing 110. In some embodiments, the second direction parallel to the central longitudinal axis 102 of the device 100 and/or the housing 110 may be opposite the first direction parallel to the central longitudinal axis 102 of the device 100 and/or the housing 110. In some embodiments, the second rounded projection 138 may be hemispherical in shape. Other configurations are also contemplated. In at least some embodiments, the first rounded projection 135 may extend to a lowermost extent below and/or downward of the lower surface 168 of the spacer plate 130. In some embodiments, the second locking element 136 may be monolithically formed with the spacer plate 130. In some embodiments, the second locking element 136 may be circumferentially spaced apart from the first locking element 133. Additional details regarding the second locking element 136 and/or its function are described herein with respect to FIG. 7. FIG. 4 illustrates selected aspects of the first iris 120 in more detail. In some embodiments, the first iris 120 may include a first circumferential ring 122 positioned coaxially relative to the central opening 112 of the housing 110 and/or the central longitudinal axis 102. In some embodiments, the first circumferential ring 122 may be positioned adjacent the housing 110. In some embodiments, the first circumferential ring 122 may be positioned at least partially within the housing 110. In some embodiments, the first iris 120 and/or the first circumferential ring 122 may have an outer perimeter and/or an outer circumference that is substantially circular.

The first iris 120 may include a first plurality of arms 124 extending radially inward from the first circumferential ring 122. The first plurality of arms 124 may define a first central opening 126 of the first iris 120. The first central opening 126 may be aligned with and/or may be positioned coaxially relative to the central opening 112 of the housing 110 and/or the central longitudinal axis 102.

In some embodiments, each arm of the first plurality of arms 124 may define a first outer hinge point 125A and a first inner hinge point 125B disposed radially inward relative to the first outer hinge point 125A. The first outer hinge point 125A of each arm of the first plurality of arms 124 may be defined by a first living hinge coupling the arm to the first circumferential ring 122. The first inner hinge point 125B of each arm of the first plurality of arms 124 may be defined by a second living hinge disposed at an opposite end of an elongated link portion 125 extending from the first living hinge to the second living hinge of each arm of the first plurality of arms 124.

The elongated link portion 125 of each arm of the first plurality of arms 124 may be coupled to and/or connected to the first circumferential ring 122 by the first living hinge disposed between the elongated link portion 125 and the first circumferential ring 122. In some embodiments, the elongated link portion 125 of each arm of the first plurality of arms 124 may be directly connected to the first circumferential ring 122 by the first living hinge. In some embodiments, the elongated link portion 125 of each arm of the first plurality of arms 124 may be fixedly attached to the first circumferential ring 122 by the first living hinge.

In some embodiments, a pivot end of each arm of the first plurality of arms 124 may be secured relative to the housing 110 and/or the spacer plate 130. In some embodiments, the pivot end of each arm of the first plurality of arms 124 may include an aperture 124B formed therein. In some embodiments, the plurality of projections 160 and/or the first plurality of projections 162 may extend through the aperture 124B and define a pivot point at and/or for the pivot end of each arm of the first plurality of arms 124. In some embodiments, the aperture 124B of each arm of the first plurality of arms 124 may be configured to receive and/or engage with the plurality of projections 160 and/or the first plurality of projections 162 extending from the spacer plate 130. In at least some embodiments, the pivot end of each arm of the first plurality of arms 124 may be configured to rotate about the plurality of projections 160 and/or the first plurality of projections 162 when the first circumferential ring 122 is rotated relative to the housing 110, the spacer plate 130, and/or the second iris 140.

A medial portion 124A of each arm of the first plurality of arms 124 may be configured to engage with medial portions of circumferentially adjacent arms of the first plurality of arms 124 to define the first central opening 126. In some embodiments, the medial portion 124A of each arm of the first plurality of arms 124 may be coupled to the second living hinge. In some embodiments, the second living hinge of each arm of the first plurality of arms 124 may couple the medial portion 124A of each arm of the first plurality of arms 124 to the elongated link portion 125 of each arm of the first plurality of arms 124. In some embodiments, the medial portion 124A of each arm of the first plurality of arms 124 may be directly connected to the elongated link portion 125 of each arm of the first plurality of arms 124 by the second living hinge of each arm of the first plurality of arms 124. In some embodiments, the first plurality of arms 124 may be integrally and/or monolithically formed with the first circumferential ring 122 from a single piece of material. In some embodiments, the first living hinge, the second living hinge, the elongated link portion 125, and the medial portion 124A of each arm of the first plurality of arms 124 may be integrally and/or monolithically formed with the first circumferential ring 122 from a single piece of material.

In at least some embodiments, the first living hinge and the second living hinge of each arm of the first plurality of arms 124 may be configured to resiliently flex, deflect, and/or bend to permit relative movement between portions of each arm of the first plurality of arms 124 and/or the first iris 120 on opposite sides of the first living hinge and/or the second living hinge. In a preferred configuration, the first iris 120 may be made from a polymeric material. Some suitable but non-limiting examples of materials that may be used to form the first iris 120, the first circumferential ring 122, the first plurality of arms 124, etc., including but not limited to metals and metal alloys, composites, ceramics, polymers, and the like, are described below.

In some embodiments, the first iris 120 may be manufactured using one or more a variety of methods. In some embodiments, the first iris 120 may be machined. In some embodiments, the first iris 120 may be cut using a waterjet. In some embodiments, the first iris 120 may be laser cut. In some embodiments, the first iris 120 may be injection molded. In some embodiments, the first iris 120 may be cast. Other methods of manufacture are also contemplated. In some embodiments, the first iris 120 and/or the first circumferential ring 122 may include one or more depressions 123 extending radially inward from the outer perimeter and/or the outer circumference. In some embodiments, the one or more depressions 123 may be provided for manufacturing reasons and may have substantially no functional effect on the first iris 120.

In some embodiments, the first iris 120 may include a first lever 121 fixedly attached to and/or extending radially outward from the first circumferential ring 122. In some embodiments, the first lever 121 may extend radially outward from the outer perimeter and/or the outer circumference of the first iris 120 and/or the first circumferential ring 122. The first lever 121 may extend radially outward along a radius extending outward from a center of the first central opening 126 and/or the central longitudinal axis 102. The first lever 121 may include a first side surface 121A facing laterally from the first radius in a first circumferential direction and a second side surface 121B disposed generally opposite the first side surface 121A, the second side surface 121B facing laterally from the first radius in a second circumferential direction generally opposite the first circumferential direction. In some embodiments, the first side surface 121A and/or the second side surface 121B of the first lever 121 may be configured to engage and/or contact the housing 110 to stop rotation of the first iris 120 and/or the first lever 121 in the first circumferential direction and the second circumferential direction, respectively.

In some embodiments, the first iris 120 may include a first open position recess 127 formed in the first circumferential ring 122, a first closed position recess 128 formed in the first circumferential ring 122, and a first guide recess 129 formed in the first circumferential ring 122. In some embodiments, the first open position recess 127, the first closed position recess 128, and the first guide recess 129 may be disposed radially inward of the outer perimeter and/or the outer circumference of the first circumferential ring 122. In some embodiments, the first open position recess 127, the first closed position recess 128, and the first guide recess 129 may be disposed within an opposing half of the first iris 120 and/or an opposing half of the first circumferential ring 122 from the first lever 121.

In some embodiments, the first open position recess 127, the first closed position recess 128, and the first guide recess 129 may extend into the first circumferential ring 122 from a lower surface of the first circumferential ring 122. In some embodiments, the first open position recess 127, the first closed position recess 128, and the first guide recess 129 may extend into the first circumferential ring 122 from the lower surface of the first circumferential ring 122 in a direction parallel to the central longitudinal axis 102 of the device 100 and/or the housing 110. In some embodiments, the first open position recess 127, the first closed position recess 128, and the first guide recess 129 may extend into the first circumferential ring 122 from the lower surface of the first circumferential ring 122 in the first direction parallel to the central longitudinal axis 102 of the device 100 and/or the housing 110. In at least some embodiments, the first open position recess 127, the first closed position recess 128, and/or the first guide recess 129 may not extend completely through the first circumferential ring 122. In some alternative embodiments, portions of the first open position recess 127, the first closed position recess 128, and/or the first guide recess 129 may extend completely through the first circumferential ring 122. In some embodiments, the first guide recess 129 may be disposed circumferentially between the first open position recess 127 and the first closed position recess 128.

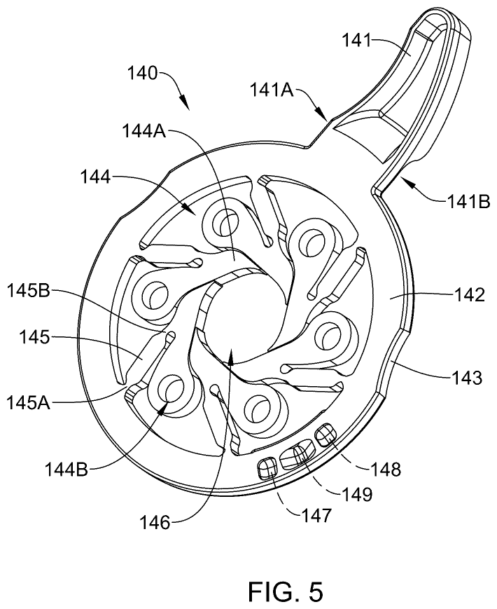

FIG. 5 illustrates selected aspects of the second iris 140 in more detail. In some embodiments, the second iris 140 may include a second circumferential ring 142 positioned coaxially relative to the central opening 112 of the housing 110 and/or the central longitudinal axis 102. In some embodiments, the second circumferential ring 142 may be positioned adjacent the housing 110. In some embodiments, the second circumferential ring 142 may be positioned at least partially within the housing 110. In some embodiments, the second iris 140 and/or the second circumferential ring 142 may have an outer perimeter and/or an outer circumference that is substantially circular.

The second iris 140 may include a second plurality of arms 144 extending radially inward from the second circumferential ring 142. The second plurality of arms 144 may define a second central opening 146 of the second iris 140. The second central opening 146 may be aligned with and/or may be positioned coaxially relative to the central opening 112 of the housing 110, the central opening 132 of the spacer plate 130, and/or the first central opening 126 of the first iris 120.

In some embodiments, each arm of the second plurality of arms 144 may define a second outer hinge point 145A and a second inner hinge point 145B disposed radially inward relative to the second outer hinge point 145A, similar to the first outer hinge point 125A and the first inner hinge point 125B. The second outer hinge point 145A of each arm of the second plurality of arms 144 may be defined by a first living hinge coupling the arm to the second circumferential ring 142. The second inner hinge point 145B of each arm of the second plurality of arms 144 may be defined by a second living hinge disposed at an opposite end of an elongated link portion 145 extending from the first living hinge to the second living hinge of each arm of the second plurality of arms 144.

The elongated link portion 145 of each arm of the second plurality of arms 144 may be coupled to and/or connected to the second circumferential ring 142 by the first living hinge disposed between the elongated link portion 145 and the second circumferential ring 142. In some embodiments, the elongated link portion 145 of each arm of the second plurality of arms 144 may be directly connected to the second circumferential ring 142 by the first living hinge. In some embodiments, the elongated link portion 145 of each arm of the second plurality of arms 144 may be fixedly attached to the second circumferential ring 142 by the first living hinge.

In some embodiments, a pivot end of each arm of the second plurality of arms 144 may be secured relative to the housing 110 and/or the spacer plate 130. In some embodiments, the pivot end of each arm of the second plurality of arms 144 may include an aperture formed therein. In some embodiments, the plurality of projections 160 and/or the second plurality of projections may extend through the aperture and engage with the housing 110 and/or the plurality of apertures 116. In some embodiments, the aperture may be configured to engage with the plurality of projections 160 and/or the second plurality of projections extending from the spacer plate 130. In at least some embodiments, the pivot end of each arm of the second plurality of arms 144 may be configured to rotate about the plurality of projections 160 and/or the second plurality of projections when the second circumferential ring 142 is rotated relative to the housing 110, the spacer plate 130, and/or the first iris 120.

A medial portion 144A of each arm of the second plurality of arms 144 may be configured to engage with medial portions of circumferentially adjacent arms of the second plurality of arms 144 to define the second central opening 146. The medial portion 144A of each arm of the second plurality of arms 144 may be coupled to the second living hinge. In some embodiments, the second living hinge of each arm of the second plurality of arms 144 may couple the medial portion 144A of each arm of the second plurality of arms 144 to the elongated link portion 145 of each arm of the second plurality of arms 144. In some embodiments, the medial portion 144A of each arm of the second plurality of arms 144 may be directly connected to the elongated link portion 145 of each arm of the second plurality of arms 144 by the second living hinge of each arm of the second plurality of arms 144. In some embodiments, the second plurality of arms 144 may be integrally and/or monolithically formed with the second circumferential ring 142 from a single piece of material. In some embodiments, the first living hinge, the second living hinge, the elongated link portion 145, and the medial portion 144A of each arm of the second plurality of arms 144 may be integrally and/or monolithically formed with the second circumferential ring 142 from a single piece of material.

In at least some embodiments, the first living hinge and the second living hinge of each arm of the second plurality of arms 144 may be configured to resiliently flex, deflect, and/or bend to permit relative movement between portions of each arm of the second plurality of arms 144 and/or the second iris 140 on opposite sides of the first living hinge and/or the second living hinge. In a preferred configuration, the second iris 140 may be made from a polymeric material. Some suitable but non-limiting examples of materials that may be used to form the second iris 140, the second circumferential ring 142, the second plurality of arms 144, etc., including but not limited to metals and metal alloys, composites, ceramics, polymers, and the like, are described below.

In some embodiments, the second iris 140 may be manufactured using one or more a variety of methods. In some embodiments, the second iris 140 may be machined. In some embodiments, the second iris 140 may be cut using a waterjet. In some embodiments, the second iris 140 may be laser cut. In some embodiments, the second iris 140 may be injection molded. In some embodiments, the second iris 140 may be cast. Other methods of manufacture are also contemplated. In some embodiments, the second iris 140 and/or the second circumferential ring 142 may include one or more depressions 143 extending radially inward from the outer perimeter and/or the outer circumference. In some embodiments, the one or more depressions 143 may be provided for manufacturing reasons and may have substantially no functional effect on the second iris 140.

In some embodiments, the second iris 140 may include a second lever 141 fixedly attached to and/or extending radially outward from the second circumferential ring 142. In some embodiments, the second lever 141 may extend radially outward from the outer perimeter and/or the outer circumference of the second iris 140 and/or the second circumferential ring 142. The second lever 141 may extend radially outward along a second radius extending outward from a center of the second central opening 146 and/or the central longitudinal axis 102. The second lever 141 may include a first side surface 141A facing laterally from the second radius in the first circumferential direction and a second side surface 141B disposed generally opposite the first side surface, the second side surface 141B facing laterally from the second radius in the second circumferential direction generally opposite the first circumferential direction. The first side surface 141A and/or the second side surface 141B of the second lever 141 may be configured to engage and/or contact the housing 110 to stop rotation of the second iris 140 and/or the second lever 141 in the first circumferential direction and the second circumferential direction, respectively.

In some embodiments, the second iris 140 may include a second open position recess 147 formed in the second circumferential ring 142, a second closed position recess 148 formed in the second circumferential ring 142, and a second guide recess 149 formed in the second circumferential ring 142. In some embodiments, the second open position recess 147, the second closed position recess 148, and the second guide recess 149 may be disposed radially inward of the outer perimeter and/or the outer circumference of the second circumferential ring 142. In some embodiments, the second open position recess 147, the second closed position recess 148, and the second guide recess 149 may be disposed within an opposing half of the second iris 140 and/or an opposing half of the second circumferential ring 142 from the second lever 141.

In some embodiments, the second open position recess 147, the second closed position recess 148, and the second guide recess 149 may extend into the second circumferential ring 142 from an upper surface of the second circumferential ring 142. In some embodiments, the second open position recess 147, the second closed position recess 148, and the second guide recess 149 may extend into the second circumferential ring 142 from the upper surface of the second circumferential ring 142 in a direction parallel to the central longitudinal axis 102 of the device 100 and/or the housing 110. In some embodiments, the second open position recess 147, the second closed position recess 148, and the second guide recess 149 may extend into the second circumferential ring 142 from the upper surface of the second circumferential ring 142 in the second direction parallel to the central longitudinal axis 102 of the device 100 and/or the housing 110. In at least some embodiments, the second open position recess 147, the second closed position recess 148, and/or the second guide recess 149 may not extend completely through the second circumferential ring 142. In some alternative embodiments, portions of the second open position recess 147, the second closed position recess 148, and/or the second guide recess 149 may extend completely through the second circumferential ring 142. In some embodiments, the second guide recess 149 may be disposed circumferentially between the second open position recess 147 and the second closed position recess 148.

FIGS. 6, 7, and 8A-8C illustrate selected aspects related to the function and/or operation of the device 100. To improve clarity, the cover plate 150 is not shown in FIGS. 6-7. Each and every reference number illustrated may not be expressly discussed (and vice versa) with respect to FIGS. 6-8C, but earlier discussion related to such reference numbers (e.g., with respect to FIGS. 1-5) may apply.

FIG. 6 illustrates the first iris 120 and/or the first plurality of arms 124 in a first configuration and/or a first open position. The first plurality of arms 124 may be configured to shift between the first configuration of the first plurality of arms 124 and a second configuration of the first plurality of arms 124 via rotation of the first circumferential ring 122 relative to the housing 110. The first lever 121 may extend radially outward from the first circumferential ring 122 through a side opening in the housing 110. The first lever 121 may be movable and/or rotatable relative to the housing 110 within the side opening of the housing 110. In some embodiments, the first plurality of arms 124 may be self-biased toward the first configuration. The first plurality of arms 124 defines the first central opening 126. In the first configuration, the first plurality of arms 124 defines a first size of the first central opening 126.

Each arm of the first plurality of arms 124 may define a pivot point at and/or within the aperture 124B (e.g., at a center of the aperture 124B). Each arm and/or the medial portion 124A of each arm of the first plurality of arms 124 may be configured to pivot around its pivot point and/or its aperture 124B. When a stent is disposed within the first central opening 126, the stent may apply a radially outward force against the medial portion 124A of each arm of the first plurality of arms 124. For the purpose of illustration, when referring to the detailed views of FIGS. 6-7, discussion will refer to a first arm of the first plurality of arms 124. It shall be understood that this discussion may apply to each arm of the first plurality of arms 124.

The radially outward force may be translated along the first arm of the first plurality of arms 124. A portion of the radially outward force may be applied along the medial portion 124A of the first arm of the first plurality of arms 124 to the pivot point at and/or within the aperture 124B as a pivot force. A portion of the radially outward force may be applied along the elongated link portion 125 and/or along a first axis extending through the first inner hinge point 125B and the first outer hinge point 125A as a link force. The first iris 120 may define a first radius extending through the central longitudinal axis 102 and the first outer hinge point 125A. The first axis may define a first angle from the first radius to the first axis. The circumferential direction of the first angle from the first radius may define and/or correspond to a circumferential force transmitted to the first circumferential ring 122. The circumferential force may urge and/or bias the first circumferential ring 122 to rotate circumferentially around the central longitudinal axis 102 independently of force applied to the first lever 121.

Similarly, while not expressly visible, the second iris 140 and/or the second plurality of arms 144 is also in a first configuration and/or in a second open position. The second plurality of arms 144 may be configured to shift between the first configuration of the second plurality of arms 144 and a second configuration of the second plurality of arms 144 via rotation of the second circumferential ring 142 relative to the housing 110. The second lever 141 may extend radially outward from the second circumferential ring 142 through the side opening in the housing 110. The second lever 141 may be movable and/or rotatable relative to the housing 110 within the side opening of the housing. In some embodiments, the second plurality of arms 144 may be self-biased toward the first configuration. The second plurality of arms 144 defines the second central opening 146. In the first configuration, the second central opening 146 has a first size. As the structures are similar, the second iris 140 may receive and/or transmit forces similarly to those of the first iris 120.

FIG. 7 illustrates the first iris 120 and/or the first plurality of arms 124 in a second configuration and/or a first closed position. Rotation of the first circumferential ring 122 of the first iris 120 relative to the housing 110 may change a size of the first central opening 126, as seen in FIGS. 6-7. In at least some embodiments, the first plurality of arms 124 may be configured to shift between the first configuration and the second configuration via rotation of the first circumferential ring 122 relative to the housing 110. In some embodiments, rotation of the first circumferential ring 122 relative to the housing 110 may be achieved by shifting and/or rotating the first lever 121 relative to the housing 110. In some embodiments, the first plurality of arms 124 may be configured to shift between the first configuration and the second configuration via clockwise rotation of the first circumferential ring 122 and/or the first lever 121 relative to the housing 110. In the second configuration, the first plurality of arms 124 defines a second size of the first central opening 126 less than the first size. Each arm of the first plurality of arms 124 may engage at least one other arm of the first plurality of arms 124 as the first plurality of arms 124 shifts from the first configuration to the second configuration. In some embodiments, the medial portion 124A of each arm of the first plurality of arms 124 may engage with the medial portion 124A of at least one other arm of the first plurality of arms 124 as the first plurality of arms 124 shifts from the first configuration to the second configuration to define the first central opening 126. In some embodiments, the medial portion 124A of each arm of the first plurality of arms 124 may engage with the medial portions of circumferentially and/or immediately adjacent arms of the first plurality of arms 124 as the first plurality of arms 124 shifts from the first configuration to the second configuration to define the first central opening 126.

Similarly, rotation of the second circumferential ring 142 of the second iris 140 relative to the housing 110 may change a size of the second central opening 146. In at least some embodiments, the second plurality of arms 144 may be configured to shift between the first configuration and a second configuration via rotation of the second circumferential ring 142 relative to the housing 110. In some embodiments, rotation of the second circumferential ring 142 relative to the housing 110 may be achieved by shifting and/or rotating the second lever 141 relative to the housing 110. In some embodiments, the second plurality of arms 144 may be configured to shift between the first configuration and the second configuration via clockwise rotation of the second circumferential ring 142 and/or the second lever 141 relative to the housing 110. In the second configuration, the second plurality of arms 144 defines a second size of the second central opening 146 less than the first size. Each arm of the second plurality of arms 144 may engage at least one other arm of the second plurality of arms 144 as the second plurality of arms 144 shifts from the first configuration to the second configuration. In some embodiments, the medial portion 144A of each arm of the second plurality of arms 144 may engage with the medial portion 144A of at least one other arm of the second plurality of arms 144 as the second plurality of arms 144 shifts from the first configuration to the second configuration to define the second central opening 146. In some embodiments, the medial portion 144A of each arm of the second plurality of arms 144 may engage with the medial portions of circumferentially and/or immediately adjacent arms of the second plurality of arms 144 as the second plurality of arms 144 shifts from the first configuration to the second configuration to define the second central opening 146.

In at least some embodiments, the first iris 120, the first lever 121, and/or the first plurality of arms 124 may be moved, shifted, and/or actuated independently of the second iris 140, the second lever 141, and/or the second plurality of arms 144. As such, at some times and/or under some circumstances, the first central opening 126 may be at the first size while the second central opening 146 is at the second size, and vice versa. In FIG. 7, both the first lever 121 of the first iris 120 and the second lever 141 of the second iris 140 have been rotated clockwise relative to the housing 110 to shift the first iris 120 and/or the first plurality of arms 124 and the second iris 140 and/or the second plurality of arms 144 toward and/or to the second configuration.

In some embodiments, the first iris 120 and/or the first plurality of arms 124 (as well as the second iris 140 and/or the second plurality of arms 144) may have a neutral position or a balanced position between the first configuration and the second configuration (and/or for the first iris 120, between the first open position and the first closed position, and for the second iris 140, between the second open position and the second closed position), wherein the first circumferential ring 122 (as well as the second circumferential ring 142) is unbiased in any particular circumferential direction. This occurs when the first radius is aligned with and/or is coaxial with the first axis, and/or when the first angle is zero. A very small circumferential force applied to the first circumferential ring 122 (or the second circumferential ring 142) in either direction may shift the first iris 120 and/or the first plurality of arms 124 (or the second iris 140 and/or the second plurality of arms 144) out of the neutral position or the balanced position, thereby reestablishing bias on the first circumferential ring 122 (or the second circumferential ring 142).

In at least some embodiments, rotation of the first circumferential ring 122 in a first circumferential direction (e.g., clockwise) may be configured to shift the first inner hinge point 125B of a first arm of the first plurality of arms 124 of the first iris 120 from a first side of the first radius extending through the central longitudinal axis 102 of the first iris 120 and/or the device 100 and the first outer hinge point 125A of the first arm of the first plurality of arms 124 of the first iris 120, as shown in FIG. 6, to a second side of the first radius extending through the central longitudinal axis 102 of the first iris 120 and/or the device 100 and the first outer hinge point 125A of the first arm of the first plurality of arms 124 of the first iris 120, as shown in FIG. 7.

As may be seen by comparing FIG. 6 to FIG. 7, rotation of the first lever 121, the first circumferential ring 122, and/or the first iris 120 in a first circumferential direction (e.g., clockwise) relative to the housing 110 may shift and/or move the first inner hinge point 125B in an opposite circumferential direction (e.g., a second circumferential direction opposite the first circumferential direction, counterclockwise) from the first circumferential ring 122. The reverse also applies, and rotation of the first lever 121, the first circumferential ring 122, and/or the first iris 120 in the second circumferential direction (e.g., counterclockwise) relative to the housing 110 may shift and/or move the first inner hinge point 125B in an opposite circumferential direction (e.g., the first circumferential direction opposite the second circumferential direction, clockwise) from the first circumferential ring 122.

The radially outward force exerted against the first arm of the first plurality of arms 124 of the first iris 120 may urge and/or bias the first circumferential ring 122 in a second circumferential direction opposite the first circumferential direction (e.g., counterclockwise) when the first inner hinge point 125B of the first arm of the first plurality of arms 124 of the first iris 120 is disposed on the first side of the first radius. When the first inner hinge point 125B of the first arm of the first plurality of arms 124 of the first iris 120 is disposed on the first side of the first radius, the first angle is negative (e.g., opens to the left or counterclockwise from the first radius). See FIG. 6 for example. As such, when the first inner hinge point 125B of the first arm of the first plurality of arms 124 of the first iris 120 is disposed on the first side of the first radius and/or when the first angle is negative, the radially outward force exerted against the first arm of the first plurality of arms 124 of the first iris 120 may urge and/or bias the first iris 120 and/or the first plurality of arms 124 towards the first configuration and/or an unlocked configuration. In some embodiments, the radially outward force exerted against the first plurality of arms 124 of the first iris 120 may urge and/or bias the first circumferential ring 122 in a first direction (e.g., the second circumferential direction, counterclockwise) in the first configuration and/or the unlocked configuration of the first plurality of arms 124.

The radially outward force exerted against the first arm of the first plurality of arms 124 of the first iris 120 may urge and/or bias the first circumferential ring 122 in the first circumferential direction (e.g., clockwise) when the first inner hinge point 125B of the first arm of the first plurality of arms 124 of the first iris 120 is disposed on the second side of the first radius. When the first inner hinge point 125B of the first arm of the first plurality of arms 124 of the first iris 120 is disposed on the first side of the first radius, the first angle is positive (e.g., opens to the right or clockwise from the first radius). See FIG. 7 for example. As such, when the first inner hinge point 125B of the first arm of the first plurality of arms 124 of the first iris 120 is disposed on the second side of the first radius and/or when the first angle is positive, the radially outward force exerted against the first arm of the first plurality of arms 124 of the first iris 120 may urge and/or bias the first iris 120 and/or the first plurality of arms 124 towards the second configuration and/or a locked configuration. In some embodiments, the radially outward force exerted against the first plurality of arms 124 of the first iris 120 may urge and/or bias the first circumferential ring 122 in a second direction (e.g., the first circumferential direction, clockwise) in the second configuration and/or the locked configuration of the first plurality of arms 124.

Clockwise rotation of the first lever 121 and/or the first circumferential ring 122 causes a radially outermost end and/or the first living hinge of each arm of the first plurality of arms 124 to shift in the clockwise direction as well. Since the pivot end of each arm of the first plurality of arms 124 is held in a fixed position relative to the housing 110 by engagement of the aperture 124B with the plurality of projections 160 and/or the first plurality of projections 162, the medial portion 124A of each arm of the first plurality of arms 124 will pivot about the plurality of projections 160 and/or the first plurality of projections 162 as the first lever 121 and/or the first circumferential ring 122 is rotated around the central longitudinal axis 102. The first living hinge and the second living hinge of each arm of the first plurality of arms 124 may resiliently flex, deflect, and/or bend at the first outer hinge point 125A and the first inner hinge point 125B, respectively, to facilitate a pivoting movement of the first plurality of arms 124 around the pivot point and/or the aperture 124B and/or the plurality of projections 160 and/or the first plurality of projections 162. As the first lever 121 and/or the first circumferential ring 122 is rotated clockwise, the pivot end of each arm of the first plurality of arms 124 may be prevented from rotation commensurately and/or equally around the central longitudinal axis 102 due to the arm being fixed in position by engagement of the aperture 124B with the plurality of projections 160 and/or the first plurality of projections 162. Lengths of the various portions of each arm of the first plurality of arms 124 remain fixed and/or constant, wherein the first living hinge and the second living hinge cooperate with the elongated link portion 125 of each arm connecting the medial portion 124A to the first circumferential ring 122 to facilitate movement and/or translation of the first plurality of arms 124 from the first configuration toward and/or to the second configuration.

In some embodiments, each arm of the first plurality of arms 124 of the first iris 120 may engage at least one other arm of the first plurality of arms 124 of the first iris 120 as the first plurality of arms 124 shifts from the first configuration of the first plurality of arms 124 to the second configuration of the first plurality of arms 124. As seen when comparing FIG. 6 to FIG. 7, for example, as the first lever 121 and/or the first circumferential ring 122 is rotated clockwise and/or as the first plurality of arms 124 shifts from the first configuration to the second configuration, the medial portion 124A of each arm of the first plurality of arms 124 may shift radially inward relative to the first circumferential ring 122 and/or toward the central longitudinal axis 102, thereby changing the size of the first central opening 126 defined by the first plurality of arms 124. In the first configuration shown in FIG. 6, the elongated link portion 125 of each arm of the first plurality of arms 124 connected to the first circumferential ring 122 by the first living hinge and/or extending between the first living hinge and the second living hinge may be oriented at an oblique angle to the first circumferential ring 122. As the first lever 121 and/or the first circumferential ring 122 is rotated clockwise toward the second configuration, the elongated link portion 125 of each arm of the first plurality of arms 124 connected to the first circumferential ring 122 by the first living hinge and/or extending between the first living hinge and the second living hinge may shift toward an angle that is closer to normal or perpendicular to the first circumferential ring 122 (e.g., wherein the first axis is aligned with the first radius), as seen in FIG. 7.

While not expressly visible in FIGS. 6 and 7, the movements and/or motions of the second iris 140 and/or the second plurality of arms 144 may be understood to be similar to or substantially the same as those of the first iris 120 and/or the first plurality of arms 124.

In some embodiments, rotation of the second circumferential ring 142 in a first circumferential direction (e.g., clockwise) may be configured to shift the second inner hinge point 145B of a first arm of the second plurality of arms 144 of the second iris 140 from a first side of a first radius extending through the central longitudinal axis 102 of the second iris 140 and/or the device 100 and the second outer hinge point 145A of the first arm of the second plurality of arms 144 of the second iris 140 to a second side of the first radius extending through the central longitudinal axis 102 of the second iris 140 and/or the device 100 and the second outer hinge point 145A of the first arm of the second plurality of arms 144 of the second iris 140.

Rotation of the second lever 141, the second circumferential ring 142, and/or the second iris 140 in a first circumferential direction (e.g., clockwise) relative to the housing 110 may shift and/or move the second inner hinge point 145B in an opposite circumferential direction (e.g., a second circumferential direction opposite the first circumferential direction, counterclockwise) from the second circumferential ring 142. The reverse also applies, and rotation of the second lever 141, the second circumferential ring 142, and/or the second iris 140 in the second circumferential direction (e.g., counterclockwise) relative to the housing 110 may shift and/or move the second inner hinge point 145B in an opposite circumferential direction (e.g., the first circumferential direction opposite the second circumferential direction, clockwise) from the second circumferential ring 142.

A radially outward force exerted against the first arm of the second plurality of arms 144 of the second iris 140 may urge and/or bias the second circumferential ring 142 in a second circumferential direction opposite the first circumferential direction (e.g., counterclockwise) when the second inner hinge point 145B of the first arm of the second plurality of arms 144 of the second iris 140 is disposed on the first side of the first radius. When the second inner hinge point 145B of the first arm of the second plurality of arms 144 of the second iris 140 is disposed on the first side of the first radius, the first angle is negative (e.g., opens to the left or counterclockwise from the first radius). As such, when the second inner hinge point 145B of the first arm of the second plurality of arms 144 of the second iris 140 is disposed on the first side of the first radius and/or when the first angle is negative, the radially outward force exerted against the first arm of the second plurality of arms 144 of the second iris 140 may urge and/or bias the second iris 140 and/or the second plurality of arms 144 towards the first configuration and/or an unlocked configuration. In some embodiments, the radially outward force exerted against the second plurality of arms 144 of the second iris 140 may urge and/or bias the second circumferential ring 142 in a first direction (e.g., the second circumferential direction, counterclockwise) in the first configuration and/or the unlocked configuration of the second plurality of arms 144.

The radially outward force exerted against the first arm of the second plurality of arms 144 of the second iris 140 may urge and/or bias the second circumferential ring 142 in the first circumferential direction (e.g., clockwise) when the second inner hinge point 145B of the first arm of the second plurality of arms 144 of the second iris 140 is disposed on the second side of the first radius. When the second inner hinge point 145B of the first arm of the second plurality of arms 144 of the second iris 140 is disposed on the first side of the first radius, the first angle is positive (e.g., opens to the right or clockwise from the first radius). As such, when the second inner hinge point 145B of the first arm of the second plurality of arms 144 of the second iris 140 is disposed on the second side of the first radius and/or when the first angle is positive, the radially outward force exerted against the first arm of the second plurality of arms 144 of the second iris 140 may urge and/or bias the second iris 140 and/or the second plurality of arms 144 towards the second configuration and/or a locked configuration. In some embodiments, the radially outward force exerted against the second plurality of arms 144 of the second iris 140 may urge and/or bias the second circumferential ring 142 in a second direction (e.g., the first circumferential direction, clockwise) in the second configuration and/or the locked configuration of the second plurality of arms 144.

Clockwise rotation of the second lever 141 and/or the second circumferential ring 142 causes a radially outermost end and/or the first living hinge of each arm of the second plurality of arms 144 to shift in the clockwise direction as well. Since the pivot end of each arm of the second plurality of arms 144 is held in a fixed position relative to the housing 110 by engagement of the aperture 144B with the plurality of projections 160 and/or the second plurality of projections, the medial portion 144A of each arm of the second plurality of arms 144 will pivot about the plurality of projections 160 and/or the second plurality of projections as the second lever 141 and/or the second circumferential ring 142 is rotated. The first living hinge and the second living hinge of each arm of the second plurality of arms 144 may resiliently flex, deflect, and/or bend at the second outer hinge point 145A and the second inner hinge point 145B, respectively, to facilitate a pivoting movement of the second plurality of arms 144 around the pivot point and/or the aperture 144B and/or the plurality of projections 160 and/or the second plurality of projections. As the second lever 141 and/or the second circumferential ring 142 is rotated clockwise, each arm of the second plurality of arms 144 may be prevented from rotation commensurately and/or equally around the central longitudinal axis 102 due to the pivot end of the arm being fixed in position by engagement of the aperture 144B with the plurality of projections 160 and/or the second plurality of projections. Lengths of the various portions of each arm of the second plurality of arms 144 remain fixed and/or constant, wherein the first living hinge and the second living hinge cooperate with the elongated link portion 145 of each arm connecting the medial portion 144A to the second circumferential ring 142 to facilitate movement and/or translation of the second plurality of arms 144 from the first configuration toward and/or to the second configuration.

In some embodiments, each arm of the second plurality of arms 144 of the second iris 140 may engage at least one other arm of the second plurality of arms 144 of the second iris 140 as the first plurality of arms 144 shifts from the first configuration of the second plurality of arms 144 to the second configuration of the second plurality of arms 144. As the second lever 141 and/or the second circumferential ring 142 is rotated clockwise and/or as the second plurality of arms 144 shifts from the first configuration to the second configuration, the medial portion 144A of each arm of the second plurality of arms 144 may shift radially inward relative to the second circumferential ring 142 and/or toward the central longitudinal axis 102, thereby changing the size of the second central opening 146 defined by the second plurality of arms 144. In the first configuration, the elongated link portion 145 of each arm of the second plurality of arms 144 connected to the second circumferential ring 142 by the first living hinge and/or extending between the first living hinge and the second living hinge may be oriented at an oblique angle to the second circumferential ring 142. As the second lever 141 and/or the second circumferential ring 142 is rotated clockwise toward the second configuration, the elongated link portion 145 of each arm of the second plurality of arms 144 connected to the second circumferential ring 142 by the first living hinge and/or extending between the first living hinge and the second living hinge may shift toward an angle that is closer to normal or perpendicular to the second circumferential ring 142 (e.g., wherein the first axis is aligned with the first radius).

By using living hinges to shift the irises and/or the pluralities of arms from the first configuration toward and/or to the second configuration (and vice versa), the complexity and high part count of traditional stent compression devices may be reduced. As such, the cost of such devices may also be reduced. Additionally, in some embodiments, the device 100 may permit a stent to be loaded into a sheath without moving or advancing the stent through the device 100 multiple times, thereby reducing the number of steps required to sheath the stent, reducing or eliminating multiple compression steps, and/or reducing opportunity for damage to the stent. In some embodiments, the device 100 may be reusable following suitable sterilization techniques. In some embodiments, the device 100 may be disposable and/or may be classified or used as a single-use device.

Additionally, it is contemplated that the device 100 may include additional irises, intervening spacer plates, etc. to accommodate a stent of longer length and/or varying outer diameter (in a first configuration and/or in a compressed configuration). For example, in some embodiments, the second size of the first central opening 126 may be the same as second size of the second central opening 146, and additional central openings of additional irises may have a second size that is the same as the second size of the first central opening 126 and/or the second central opening 146. In some embodiments, the second size of the first central opening 126 may be different from the second size of the second central opening 146, and additional central openings of additional irises may have a second size that is the same as the second size of the first central opening 126 and the second central opening 146, the additional central openings of additional irises may have a second size that is the same as the second size of one of the first central opening 126 and the second central opening 146, or the additional central openings of additional irises may have a second size that is the different from the second size of the first central opening 126 and the second central opening 146. Other configurations are also contemplated.

In some embodiments, the first iris 120 and/or the medial portion(s) 124A of the first plurality of arms 124, the second iris 140 and/or the medial portion(s) 144A of the second plurality of arms 144, and/or additional irises and/or medial portions of the pluralities of arms thereof could be shaped or tapered axially to accommodate stents having outer surfaces that are curved or tapered in an axial direction.

In some embodiments, the first iris 120 may have a first axial thickness and the second iris 140 may have a second axial thickness. In some embodiments, the first axial thickness may be equal to the second axial thickness. In some embodiments, the first axial thickness may be different from the second axial thickness. In some embodiments, the first axial thickness may be less than the second axial thickness. In some embodiments, the first axial thickness may be greater than the second axial thickness. In some embodiments, additional irises may each have an axial thickness. The axial thickness of any additional iris may be equal to, more than, or less than the first axial thickness and/or the second axial thickness, as desired, to accommodate stents having different sizes, different lengths, and/or different radial compression requirements along their length.

In some embodiments, the first locking element 133 and/or the first deflectable tab 134 may be disposed radially inward of the outer perimeter and/or the outer circumference of the first circumferential ring 122, as seen in FIGS. 6-7. In some embodiments, a radially outermost extent of the first locking element 133 and/or the first deflectable tab 134 may be disposed radially inward of the outer perimeter and/or the outer circumference of the first circumferential ring 122. Similarly, the second locking element 136 and/or the second deflectable tab 137 may be disposed radially inward of the outer perimeter and/or the outer circumference of the second circumferential ring 142. In some embodiments, a radially outermost extent of the second locking element 136 and/or the second deflectable tab 137 may be disposed radially inward of the outer perimeter and/or the outer circumference of the second circumferential ring 142.

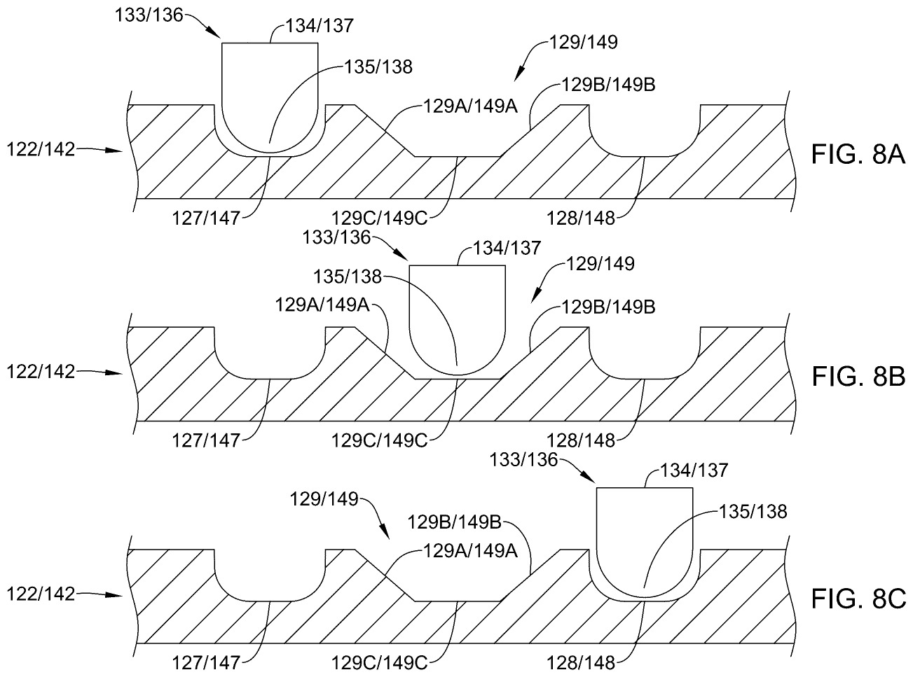

In some embodiments, the first locking element 133 may be configured to selectively engage with the first open position recess 127, the first closed position recess 128, and the first guide recess 129, as seen in FIGS. 8A-8C. In some embodiments, the first locking element 133 may be engaged with the first iris 120 to selectively lock the first iris 120 in the first open position (e.g., the first configuration) and the first closed position (e.g., the second configuration), such that the first iris 120 is unable to rotate relative to the housing 110 on its own and/or without additional force applied to the first lever 121. Similarly, in some embodiments, the second locking element 136 may be configured to selectively engage with the second open position recess 147, the second closed position recess 148, and the second guide recess 149, as seen in FIGS. 8A-8C. In some embodiments, the second locking element 136 may be engaged with the second iris 140 to selectively lock the second iris 140 in the second open position (e.g., the first configuration) and the second closed position (e.g., the second configuration), such that the second iris 140 is unable to rotate relative to the housing 110 on its own and/or without additional force applied to the second lever 141.

In some embodiments, the first rounded projection 135 may be configured to extend into the first open position recess 127 when the first iris 120 is disposed in the first open position (e.g., the first configuration), as seen in FIGS. 6 and 8A. In some embodiments, the first rounded projection 135 may be configured to extend into the first closed position recess 128 when the first iris 120 is disposed in the first closed position (e.g., the second configuration), as seen in FIGS. 7 and 8C. Similarly, the second rounded projection 138 may be configured to extend into the second open position recess 147 when the second iris 140 is disposed in the second open position (e.g., the first configuration), as seen in FIG. 8A. In some embodiments, the second rounded projection 138 may be configured to extend into the second closed position recess 148 when the second iris 140 is disposed in the second closed position (e.g., the second configuration), as seen in FIG. 8C.

When shifting the first iris 120 from the first open position (e.g., the first configuration) to the first closed position (e.g., the second configuration), such as when compressing a stent as described herein, the first rounded projection 135 may be moved from the first open position recess 127 (e.g., FIG. 8A) to the first guide recess 129 (e.g., FIG. 8B) to the first closed position recess 128 (e.g., FIG. 8C). When shifting the first iris 120 from the first closed position (e.g., the second configuration) to the first open position (e.g., the first configuration), such as when loading a stent into a sheath as described herein, the first rounded projection 135 may be moved from the first closed position recess 128 (e.g., FIG. 8C) to the first guide recess 129 (e.g., FIG. 8B) to the first open position recess 127 (e.g., FIG. 8A).

As seen in FIGS. 8A-8C, the first guide recess 129 may include a first open position ramp 129A, a first closed position ramp 129B, and a first central recess 129C disposed between the first open position ramp 129A and the first closed position ramp 129B. In some embodiments, the first open position recess 127 may have sides and/or sidewalls that are substantially parallel to the central longitudinal axis 102 (not shown but extends vertically on the drawing sheet). In some embodiments, the first closed position recess 128 may have sides and/or sidewalls that are substantially parallel to the central longitudinal axis 102.

In some embodiments, the first open position ramp 129A and the first closed position ramp 129B may be configured to reduce the amount of force required to move the first rounded projection 135 out of the first guide recess 129 and/or the first central recess 129C, thereby making it easier to move the first iris 120 into the first open position and/or the first closed position than out of the first open position and/or the first closed position.

In at least some embodiments, the first open position ramp 129A may be formed at an oblique angle to the central longitudinal axis 102. In at least some embodiments, the first closed position ramp 129B may be formed at an oblique angle to the central longitudinal axis 102. In some embodiments, the oblique angle of the first open position ramp 129A may be equal to the oblique angle of the first closed position ramp 129B. In at least some embodiments, the first open position ramp 129A may be formed at an acute angle to the central longitudinal axis 102. In at least some embodiments, the first closed position ramp 129B may be formed at an acute angle to the central longitudinal axis 102. In some embodiments, the acute angle of the first open position ramp 129A may be equal to the acute angle of the first closed position ramp 129B. Other configurations are also contemplated. In some alternative embodiments, the first open position ramp 129A and/or the first closed position ramp 129B may be curved. In some embodiments, the first open position ramp 129A and/or the first closed position ramp 129B may be convex. In some embodiments, the first open position ramp 129A and/or the first closed position ramp 129B may be concave.

In some embodiments, more force may be required to move the first rounded projection 135 from the first open position recess 127 to the first guide recess 129 than from the first guide recess 129 to the first closed position recess 128. In some embodiments, more force may be required to move the first rounded projection 135 from the first open position recess 127 to the first guide recess 129 than from the first guide recess 129 to the first open position recess 127. In some embodiments, more force may be required to move the first rounded projection 135 from the first closed position recess 128 to the first guide recess 129 than from the first guide recess 129 to the first open position recess 127. In some embodiments, more force may be required to move the first rounded projection 135 from the first closed position recess 128 to the first guide recess 129 than from the first guide recess 129 to the first closed position recess 128. In some embodiments, more force is required to unlock the first iris 120 from the first open position than is required to lock the first iris 120 in the first open position. In some embodiments, more force is required to unlock the first iris 120 from the first closed position than is required to lock the first iris 120 in the first closed position.

In at least some embodiments, the first locking element 133, the first open position recess 127, the first closed position recess 128, and the first guide recess 129 may be positioned relative to the first lever 121 and/or the side opening in the housing 110 such that the first iris 120 and/or the first circumferential ring 122 is non-deflectable and/or non-deformable in an axial direction of the device 100, thereby preventing deflection and/or deformation of the first iris 120 from reducing the force required to move the first rounded projection 135 from the first open position recess 127 to the first guide recess 129 and/or to move the first rounded projection 135 from the first closed position recess 128 to the first guide recess 129. Instead, the force required to move the first rounded projection 135 from the first open position recess 127 to the first guide recess 129 and/or to move the first rounded projection 135 from the first closed position recess 128 to the first guide recess 129 may be determined by properties of the first deflectable tab 134. In some embodiments, the force required to move the first rounded projection 135 from the first open position recess 127 to the first guide recess 129 and/or to move the first rounded projection 135 from the first closed position recess 128 to the first guide recess 129 may be changed by changing the thickness of the first deflectable tab 134 in the axial direction of the device 100.

When shifting the second iris 140 from the second open position (e.g., the first configuration) to the second closed position (e.g., the second configuration), such as when compressing a stent as described herein, the second rounded projection 138 may be moved from the second open position recess 147 (e.g., FIG. 8A) to the second guide recess 149 (e.g., FIG. 8B) to the second closed position recess 148 (e.g., FIG. 8C). When shifting the second iris 140 from the second closed position (e.g., the second configuration) to the second open position (e.g., the first configuration), such as when loading a stent into a sheath as described herein, the second rounded projection 138 may be moved from the second closed position recess 148 (e.g., FIG. 8C) to the second guide recess 149 (e.g., FIG. 8B) to the second open position recess 147 (e.g., FIG. 8A).

As seen in FIGS. 8A-8C, the second guide recess 149 may include a second open position ramp 149A, a second closed position ramp 149B, and a second central recess 149C disposed between the second open position ramp 149A and the second closed position ramp 149B. In some embodiments, the second open position recess 147 may have sides and/or sidewalls that are substantially parallel to the central longitudinal axis 102 (not shown but extends vertically on the drawing sheet). In some embodiments, the second closed position recess 148 may have sides and/or sidewalls that are substantially parallel to the central longitudinal axis 102.

In some embodiments, the second open position ramp 149A and the second closed position ramp 149B may be configured to reduce the amount of force required to move the second rounded projection 138 out of the second guide recess 149 and/or the second central recess 149C, thereby making it easier to move the second iris 140 into the second open position and/or the second closed position than out of the second open position and/or the second closed position.

In at least some embodiments, the second open position ramp 149A may be formed at an oblique angle to the central longitudinal axis 102. In at least some embodiments, the second closed position ramp 149B may be formed at an oblique angle to the central longitudinal axis 102. In some embodiments, the oblique angle of the second open position ramp 149A may be equal to the oblique angle of the second closed position ramp 149B. In at least some embodiments, the second open position ramp 149A may be formed at an acute angle to the central longitudinal axis 102. In at least some embodiments, the second closed position ramp 149B may be formed at an acute angle to the central longitudinal axis 102. In some embodiments, the acute angle of the second open position ramp 149A may be equal to the acute angle of the second closed position ramp 149B. Other configurations are also contemplated. In some alternative embodiments, the second open position ramp 149A and/or the second closed position ramp 149B may be curved. In some embodiments, the second open position ramp 149A and/or the second closed position ramp 149B may be convex. In some embodiments, the second open position ramp 149A and/or the second closed position ramp 149B may be concave.

In some embodiments, more force may be required to move the second rounded projection 138 from the second open position recess 147 to the second guide recess 149 than from the second guide recess 149 to the second closed position recess 148. In some embodiments, more force may be required to move the second rounded projection 138 from the second open position recess 147 to the second guide recess 149 than from the second guide recess 149 to the second open position recess 147. In some embodiments, more force may be required to move the second rounded projection 138 from the second closed position recess 148 to the second guide recess 149 than from the second guide recess 149 to the second open position recess 147. In some embodiments, more force may be required to move the second rounded projection 138 from the second closed position recess 148 to the second guide recess 149 than from the second guide recess 149 to the second closed position recess 148. In some embodiments, more force is required to unlock the second iris 140 from the second open position than is required to lock the second iris 140 in the second open position. In some embodiments, more force is required to unlock the second iris 140 from the second closed position than is required to lock the second iris 140 in the second closed position.

In at least some embodiments, the second locking element 136, the second open position recess 147, the second closed position recess 148, and the second guide recess 149 may be positioned relative to the second lever 141 and/or the side opening in the housing 110 such that the second iris 140 and/or the second circumferential ring 142 is non-deflectable and/or non-deformable in an axial direction of the device 100, thereby preventing deflection and/or deformation of the second iris 140 from reducing the force required to move the second rounded projection 138 from the second open position recess 147 to the second guide recess 149 and/or to move the second rounded projection 138 from the second closed position recess 148 to the second guide recess 149. Instead, the force required to move the second rounded projection 138 from the second open position recess 147 to the second guide recess 149 and/or to move the second rounded projection 138 from the second closed position recess 148 to the second guide recess 149 may be determined by properties of the second deflectable tab 137. In some embodiments, the force required to move the second rounded projection 138 from the second open position recess 147 to the second guide recess 149 and/or to move the second rounded projection 138 from the second closed position recess 148 to the second guide recess 149 may be changed by changing the thickness of the second deflectable tab 137 in the axial direction of the device 100.

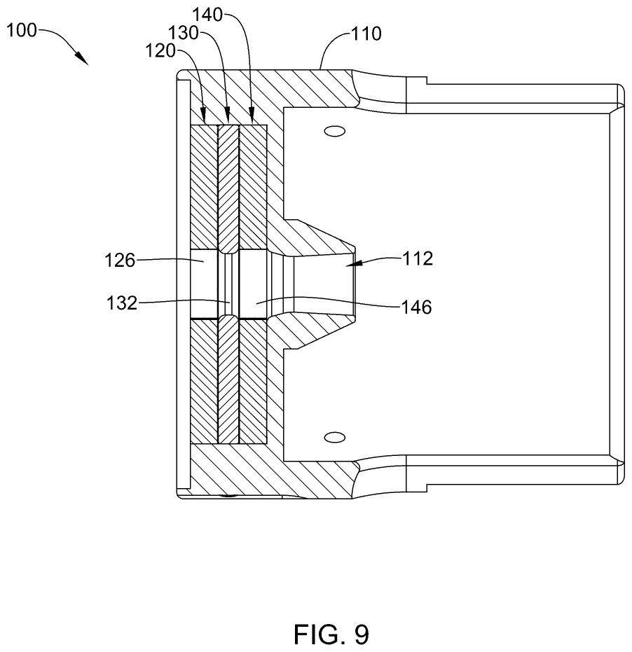

FIGS. 9-13 are partial schematic cross-sectional views of the device 100 showing selected aspects of a method of radially compressing a stent 200 and/or a method of loading a stent 200 into a sheath 300. The device 100 may generally include elements and/or features as described herein. For improved clarity and understanding, some elements or features of the device 100 are not shown, are not shown in their entirety, or are shown schematically.

FIG. 9 illustrates a partial schematic cross-sectional view of the device 100 for radially compressing a stent 200 as described herein. In at least some embodiments, the device 100 may be used to load the stent 200 into a sheath 300. The device 100 may include the housing 110 defining the central opening 112. In some embodiments, at least a portion of the central opening 112 may have an inner diameter that is tapered radially outward in an axial direction. In some embodiments, the central opening 112 may have an inner diameter that is generally constant. In some embodiments, the central opening 112 may have an inner diameter that is generally constant along a portion of its axial length and tapered along a different portion of its axial length. Other configurations are also contemplated.

The device 100 may comprise the first iris 120 including the first circumferential ring 122 and the first plurality of arms 124 (e.g., FIG. 2) extending radially inward from the first circumferential ring 122 to define the first central opening 126. The first central opening 126 may have a first size in a first configuration of the first plurality of arms 124 and a second size in a second configuration of the first plurality of arms 124. The device 100 may include the spacer plate 130 including and/or defining the central opening 132 therein. In some embodiments, the central opening 132 may have a generally fixed size. The device 100 may include the second iris 140 including the second plurality of arms 144 (e.g., FIG. 2) defining the second central opening 146. The second central opening 146 may have a first size in a first configuration of the second plurality of arms 144 and a second size in a second configuration of the second plurality of arms 144.

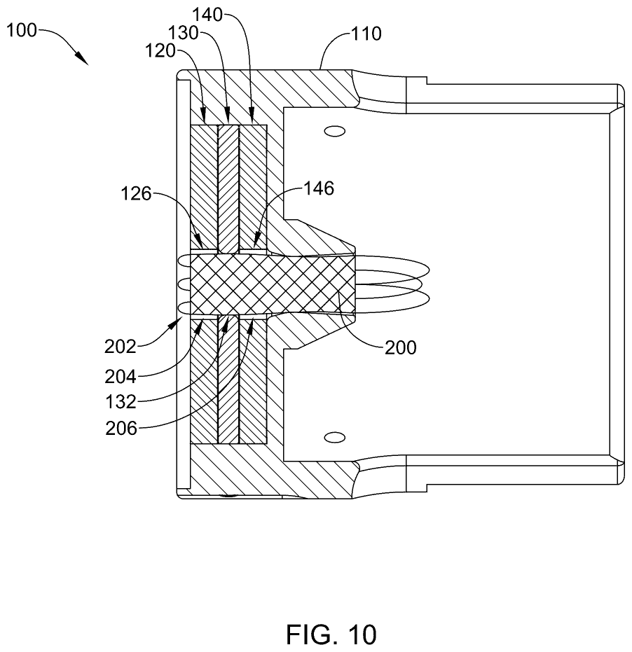

A method of radially compressing the stent 200 and/or a method of loading the stent 200 into the sheath 300 may comprise inserting a stent 200 in a first configuration into the first iris 120 such that a first end portion 202 of the stent 200 is disposed outside of the first iris 120, as seen in FIG. 10. In some embodiments, the method may comprise inserting the stent 200 in the first configuration into the first central opening 126 of the first iris 120 disposed in the first configuration and/or the first open position of the first plurality of arms 124 such that a first portion 204 of the stent 200 is disposed within the first iris 120. In the first configuration and/or the first open position of the first plurality of arms 124, the first locking element 133 and/or the first rounded projection 135 may be engaged with and/or disposed within the first open position recess 127 (e.g., FIGS. 6, 8A).

In at least some embodiments, the first portion 204 of the stent 200 is disposed immediately adjacent the first end portion 202 of the stent 200. In some embodiments, in the first configuration, the stent 200 may be in an expanded configuration. In some embodiments, in the first configuration, the stent 200 may be in a partially collapsed state. For example, in some embodiments, the device 100 may include a loading funnel (not shown) adapted to partially collapse the stent 200 as the stent 200 is inserted into the first central opening 126 of the first iris 120 and/or the central opening 112 of the housing 110. In some embodiments, the loading funnel may be configured to releasably attach to the housing 110. For example, in the view shown in FIG. 10, the loading funnel may be configured to be inserted into the housing 110 from the right side of the view until at least a portion of the loading funnel is positioned adjacent to and/or is engaged with the housing 110 proximate the central opening 112. Other configurations are also contemplated. In some embodiments, the method may include inserting the stent 200 into and/or through the central opening 112 of the housing 110 before inserting the stent 200 in the first configuration into first central opening 126 of the first iris 120.

In some embodiments, the method may comprise inserting the stent 200 in the first configuration into the first iris 120 and a second iris 140 axially offset from the first iris 120 and/or a second central opening 146 of the second iris 140 axially offset from the first iris 120 such that a second portion 206 of the stent 200 is disposed within the second iris 140. As discussed herein, the second iris 140 may include a second circumferential ring 142 and a second plurality of arms 144 extending radially inward from the second circumferential ring 142 to define the second central opening 146. In some embodiments, the method may comprise inserting the stent 200 in the first configuration into the second central opening 146 of the second iris 140 disposed in the first configuration and/or the second open position of the second plurality of arms 144 such that a second portion 206 of the stent 200 is disposed within the second iris 140. In the first configuration and/or the second open position of the second plurality of arms 144, the second locking element 136 and/or the second rounded projection 138 may be engaged with and/or disposed within the second open position recess 147 (e.g., FIG. 8C).

In some embodiments including the second iris 140, the method may include inserting the stent 200 in the first configuration into and/or through the central opening 132 of the spacer plate 130. Additionally, while not expressly illustrated, the method may include inserting the stent 200 into and/or through the cover plate opening 156 of the cover plate 150 (e.g., FIG. 2).

The stent 200 may include an expandable framework defining a central lumen which, in some embodiments, may be substantially cylindrical. In some embodiments, the expandable framework may have a substantially circular cross-section. In some embodiments, the expandable framework can have a non-circular (e.g., D-shaped, elliptical, etc.) cross-section. Some suitable but non-limiting examples of materials that may be used to form the expandable framework, including but not limited to metals and metal alloys, composites, ceramics, polymers, and the like, are described below.

The stent 200 and/or the expandable framework may be configured to shift from a collapsed configuration to an expanded configuration. In some embodiments, the expandable framework may be self-expanding. In some embodiments, the expandable framework may be self-biased toward the expanded configuration. In some embodiments, the expandable framework may be mechanically expandable. In some embodiments, the expandable framework may be balloon expandable. Other configurations are also contemplated.

In some embodiments, the stent 200 may be a part of a replacement heart valve implant. It will be appreciated that the replacement heart valve implant can be any type of heart valve implant (e.g., a mitral valve implant, an aortic valve implant, etc.). The replacement heart valve implant can be configured to allow one-way flow through the replacement heart valve implant from an inflow end to an outflow end. In some embodiments of a replacement heart valve implant, the stent 200 and/or the expandable framework may define a lower crown proximate an inflow end of the replacement heart valve implant, an upper crown proximate an outflow end of the replacement heart valve implant, and a plurality of stabilization arches extending downstream from the outflow end.

In some embodiments, the replacement heart valve implant may include a plurality of valve leaflets disposed within the central lumen. The plurality of valve leaflets may be coupled, secured, and/or fixedly attached to the stent 200 and/or the expandable framework. In some embodiments, the plurality of valve leaflets can be monolithically formed with each other, such that the plurality of valve leaflets is formed as a single unitary and/or monolithic unit. In some embodiments, the plurality of valve leaflets may be formed monolithically with other structures such as an inner skirt and/or an outer skirt, base structures, liners, or the like. The plurality of valve leaflets may be configured to substantially restrict fluid from flowing through the replacement heart valve implant in a closed position. For example, in some embodiments, free edges of the plurality of valve leaflets may move into coaptation with one another in the closed position to substantially restrict fluid from flowing through the replacement heart valve implant. The free edges of the plurality of valve leaflets may move apart from each other in an open position to permit fluid flow through the replacement heart valve implant.

In some embodiments, the replacement heart valve implant may include an inner skirt. The inner skirt may be disposed on and/or extend along an inner surface of the stent 200 and/or the expandable framework. In at least some embodiments, the inner skirt may be fixedly attached to the stent 200 and/or the expandable framework. The inner skirt may direct fluid, such as blood, flowing through the replacement heart valve implant toward the plurality of valve leaflets. In at least some embodiments, the inner skirt may be fixedly attached to and/or monolithically formed with the plurality of valve leaflets. The inner skirt may ensure the fluid flows through the central lumen and does not flow around the plurality of valve leaflets when they are in the closed position.

In some embodiments, the replacement heart valve implant may include an outer skirt. In some embodiments, the outer skirt may be disposed on and/or extend along an outer surface of the stent 200 and/or the expandable framework. In some embodiments, the outer skirt may be disposed between the stent 200 and/or the expandable framework and native tissue in order to prevent fluid, such as blood, flowing around the stent 200 and/or the expandable framework in a downstream direction so as to ensure that the plurality of valve leaflets can stop the flow of fluid when in the closed position.

In some embodiments, the plurality of valve leaflets may be comprised of a polymer, such as a thermoplastic polymer. In some embodiments, the plurality of valve leaflets may include at least 50 percent by weight of a polymer. In some embodiments, the plurality of valve leaflets may be formed from porcine pericardium, bovine pericardium, or other tissue. Other configurations and/or materials are also contemplated.

In some embodiments, the inner skirt and/or the outer skirt may include a polymer, such as a thermoplastic polymer. In some embodiments, the inner skirt and/or the outer skirt may include at least 50 percent by weight of a polymer. In some embodiments one or more of the plurality of valve leaflets, the inner skirt, and/or the outer skirt may be formed of the same polymer or polymers. In some embodiments, the polymer may be a polyurethane.

In some embodiments, the inner skirt and/or the outer skirt may be substantially impervious to fluid. In some embodiments, the inner skirt and/or the outer skirt may be formed from a thin tissue (e.g., porcine pericardium, bovine pericardium, etc.). In some embodiments, the inner skirt and/or the outer skirt may be formed from a coated fabric material. In some embodiments, the inner skirt and/or the outer skirt may be formed from a nonporous and/or impermeable fabric material. Other configurations are also contemplated. Some suitable but non-limiting examples of materials that may be used to form the inner skirt and/or the outer skirt including but not limited to polymers, composites, and the like, are described below.

In some embodiments, the stent 200 and/or the replacement heart valve implant may have an outer extent of about 23 millimeters (mm), about 25 mm, about 27 mm, about 30 mm, etc. in an unconstrained configuration (e.g., in the expanded configuration). In some embodiments, the stent 200 and/or the replacement heart valve implant may have an outer extent of about 10 mm, about 9 mm about 8 mm, about 7 mm, about 6 mm, etc. in the collapsed configuration. Other configurations are also contemplated.

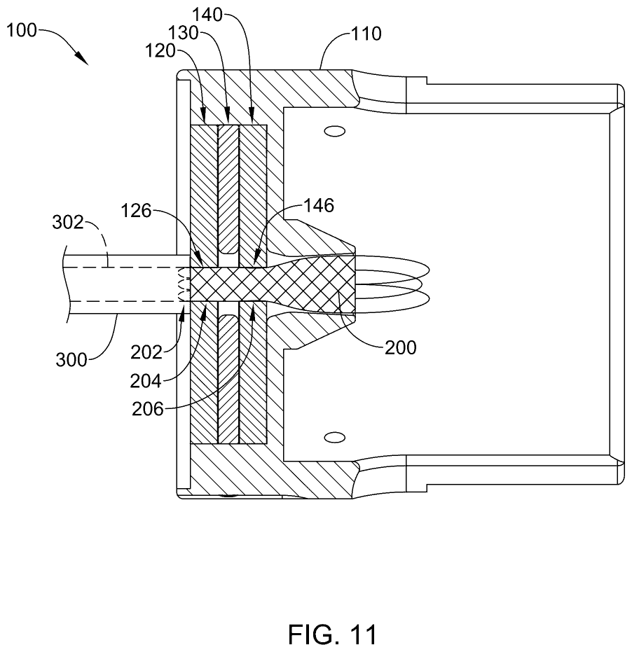

In some embodiments, the method may comprise rotating the first circumferential ring 122 of the first iris 120 relative to the housing 110 disposed about the first circumferential ring 122 to shift the first plurality of arms 124 from the first configuration and/or the first open position to the second configuration and/or the first closed position, as seen in FIG. 11, wherein the first central opening 126 has a first size in the first configuration and/or the first open position (e.g., FIG. 10) and a second size in the second configuration and/or the first closed position less than the first size (e.g., FIG. 11). In some embodiments, the method may comprise rotating the first circumferential ring 122 of the first iris 120 clockwise relative to the housing 110 disposed about the first circumferential ring 122 to shift the first plurality of arms 124 from the first configuration and/or the first open position to the second configuration and/or the first closed position. In some embodiments, rotating the first circumferential ring 122 to shift the first plurality of arms 124 from the first configuration and/or the first open position to the second configuration and/or the first closed position may move and/or shift the first rounded projection 135 from the first open position recess 127 through the first guide recess 129 to the first closed position recess 128 (e.g., FIGS. 8A-8C).

In the second configuration and/or the first closed position of the first plurality of arms 124, the first portion 204 of the stent 200 disposed within the first iris 120 and/or the first central opening 126 may be radially compressed and exert a radially outward force against the first plurality of arms 124. In at least some embodiments, in the second configuration and/or the first closed position of the first plurality of arms 124, the radially outward force against the first plurality of arms 124 may urge and/or bias the first plurality of arms 124 toward the second configuration and/or the first closed position of the first plurality of arms 124. In the second configuration and/or the first closed position of the first plurality of arms 124, the first locking element 133 and/or the first rounded projection 135 may be engaged with and/or disposed within the first closed position recess 128 (e.g., FIGS. 7, 8C).

In some embodiments, the method may comprise rotating the second circumferential ring 142 of the second iris 140 relative to the housing 110 to shift the second plurality of arms 144 from a first configuration and/or the second open position to a second configuration and/or a second closed position, wherein the second central opening 146 has a first size in the first configuration and/or the second open position and a second size in the second configuration and/or the second closed position less than the first size. In some embodiments, the method may comprise rotating the second circumferential ring 142 of the second iris 140 clockwise relative to the housing 110 to shift the second plurality of arms 144 from the first configuration and/or the second open position to the second configuration and/or the second closed position. In some embodiments, rotating the second circumferential ring 142 to shift the second plurality of arms 144 from the first configuration and/or the second open position to the second configuration and/or the second closed position may move and/or shift the second rounded projection 138 from the second open position recess 147 through the second guide recess 149 to the second closed position recess 148 (e.g., FIGS. 8A-8C).

In the second configuration and/or the second closed position of the second plurality of arms 144, the second portion 206 of the stent 200 disposed within the second iris 140 and/or the second central opening 146 may be radially compressed and exert a radially outward force against the second plurality of arms 144. In at least some embodiments, in the second configuration and/or the second closed position of the second plurality of arms 144, the radially outward force against the second plurality of arms 144 may urge and/or bias the second plurality of arms 144 toward the second configuration and/or the second closed position of the second plurality of arms 144. In the second configuration and/or the second closed position of the second plurality of arms 144, the second locking element 136 and/or the second rounded projection 138 may be engaged with and/or disposed within the second closed position recess 148 (e.g., FIG. 8C).

In some embodiments, the method may comprise positioning a sheath 300 proximate the first iris 120 with the first plurality of arms 124 in the second configuration and/or the first closed position and the first portion of the stent 200 radially compressed within the first iris 120 such that the first end portion 202 of the stent 200 is disposed within a lumen 302 of the sheath 300, as seen in FIG. 11. After positioning the sheath 300 proximate the first iris 120, the method may comprise rotating the first circumferential ring 122 of the first iris 120 relative to the housing 110 to shift the first plurality of arms 124 from the second configuration and/or the first closed position to the first configuration and/or the first open position. In some embodiments, the method may comprise rotating the first circumferential ring 122 of the first iris 120 counterclockwise relative to the housing 110 to shift the first plurality of arms 124 from the second configuration and/or the first closed position to the first configuration and/or the first open position. In some embodiments, rotating the first circumferential ring 122 to shift the first plurality of arms 124 from the second configuration and/or the first closed position to the first configuration and/or the first open position may move and/or shift the first rounded projection 135 from the first closed position recess 128 through the first guide recess 129 to the first open position recess 127 (e.g., FIGS. 8A-8C).

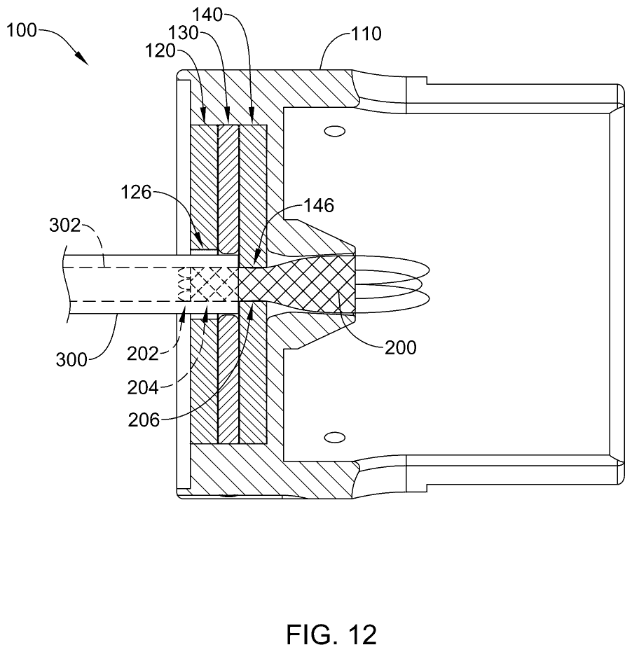

The method may further comprise moving the sheath 300 into the first iris 120 over the stent 200 such that the first portion 204 of the stent 200 that was disposed within the first iris 120 is disposed within the lumen 302 of the sheath 300, as seen in FIG. 12. In some embodiments, the sheath 300 has an inner diameter less than an outer diameter of the first portion 204 of the stent 200 when the stent 200 is in the first configuration. As such, radial compression of the stent 200 is required in order to move the stent 200 into the lumen 302 of the sheath 300.

In some embodiments, after moving the sheath 300 into the first iris 120 over the stent 200 such that the first portion 204 of the stent 200 that was disposed within the first iris 120 is disposed within the lumen 302 of the sheath 300, the method may further comprise rotating the second circumferential ring 142 of the second iris 140 relative to the housing 110 to shift the second plurality of arms 144 of the second iris 140 from the second configuration and/or the second closed position to the first configuration and/or the second open position. In some embodiments, the method may comprise rotating the second circumferential ring 142 of the second iris 140 counterclockwise relative to the housing 110 to shift the second plurality of arms 144 of the second iris 140 from the second configuration and/or the second closed position to the first configuration and/or the second open position. In some embodiments, rotating the second circumferential ring 142 to shift the second plurality of arms 144 from the second configuration and/or the second closed position to the first configuration and/or the second open position may move and/or shift the second rounded projection 138 from the second closed position recess 148 through the second guide recess 149 to the second open position recess 147 (e.g., FIGS. 8A-8C).

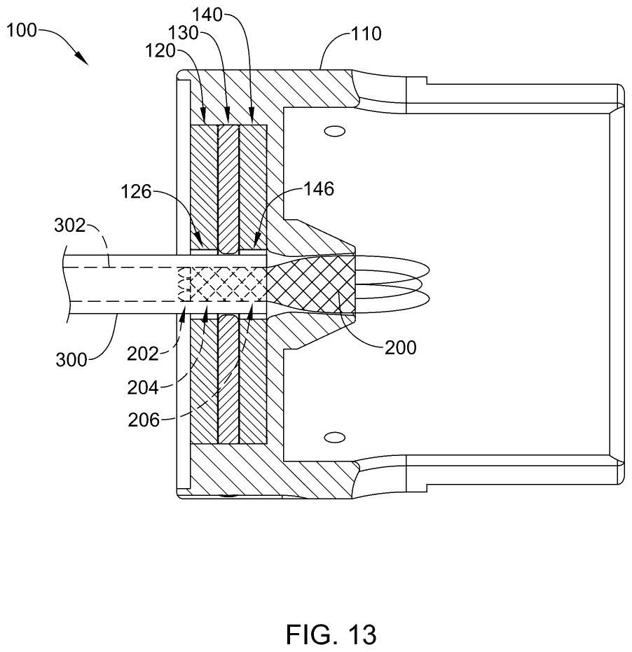

The method may further comprise moving the sheath 300 into the second iris 140 over the stent 200 in the compressed configuration such that the second portion 206 of the stent 200 that was disposed within the second iris 140 is disposed within the lumen 302 of the sheath 300, as seen in FIG. 13.

In some embodiments, wherein the device 100 includes additional irises, the process described above with respect to moving the sheath 300 into the first iris 120 and the second iris 140 may be repeated as necessary to move the sheath 300 over additional portions of the stent 200 disposed within those irises.

In some embodiments, if additional length of the stent 200 needs to be moved into the sheath 300, the stent 200 may be advanced through the central opening 112 of the housing 110 and the first iris 120 and the second iris 140 may be again shifted from the first configuration to the second configuration and the process may be repeated.

In some embodiments, after moving the sheath 300 into the second iris 140 over the stent 200 such that the second portion 206 of the stent 200 that was disposed within the second iris 140 is disposed within the lumen of the sheath 300, the sheath 300 and the first and second portions of the stent 200 that were disposed within the first and second irises, respectively, may be advanced through the central opening 112 of the housing 110, and a separate sheath may be translated over an uncovered portion of the stent 200 toward the sheath 300 to cover at least some of the uncovered portion of the stent 200 that remains outside of the sheath 300. Other configurations are also contemplated.

The materials that can be used for the various components of the device and the various elements thereof disclosed herein may include those commonly associated with medical devices and devices used and/or associated with medical devices. For simplicity purposes, the following discussion refers to the system. However, this is not intended to limit the devices and methods described herein, as the discussion may be applied to other elements, members, components, or devices disclosed herein, such as, but not limited to, the stent, the expandable framework, the first and/or second iris, the first and/or second plurality of arms, the first and/or second circumferential ring, the spacer plate, the cover plate, the housing, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A device for radially compressing a stent, comprising:
a housing including a central opening; and
a first iris positioned adjacent the housing;
wherein the first iris includes a first circumferential ring positioned coaxially relative to the central opening, a first plurality of arms extending radially inward from the first circumferential ring, and a first lever extending radially outward from the first circumferential ring;
wherein the first iris includes a first open position recess formed in the first circumferential ring, a first closed position recess formed in the first circumferential ring, and a first guide recess formed in the first circumferential ring.

2. The device of claim 1, wherein the first guide recess is disposed circumferentially between the first open position recess and the first closed position recess.

3. The device of claim 2, wherein the first guide recess includes a first open position ramp, a first closed position ramp, and a first central recess disposed between the first open position ramp and the first closed position ramp.

4. The device of claim 1, wherein a first locking element is configured to selectively engage with the first open position recess, the first closed position recess, and the first guide recess.

5. The device of claim 4, wherein the first locking element includes a first deflectable tab extending radially relative to the central opening and a first rounded projection extending from the first deflectable tab, wherein the first deflectable tab is disposed radially inward of an outer circumference of the first circumferential ring.

6. The device of claim 5, wherein the first rounded projection is configured to extend into the first open position recess when the first iris is disposed in a first open position.

7. The device of claim 6, wherein more force is required to move the first rounded projection from the first open position recess to the first guide recess than from the first guide recess to the first open position recess.

8. The device of claim 5, wherein the first rounded projection is configured to extend into the first closed position recess when the first iris is disposed in a first closed position.

9. The device of claim 8, wherein more force is required to move the first rounded projection from the first closed position recess to the first guide recess than from the first guide recess to the first closed position recess.

10. The device of claim 1, wherein the first open position recess, the first closed position recess, and the first guide recess extend into the first circumferential ring in a direction parallel to a central longitudinal axis of the housing.

11. The device of claim 1, wherein the first open position recess, the first closed position recess, and the first guide recess are disposed radially inward of an outer circumference of the first circumferential ring.

12. The device of claim 11, wherein the first open position recess, the first closed position recess, and the first guide recess are disposed within an opposing half of the first iris from the first lever.

13. A device for radially compressing a stent, comprising:
a housing including a central opening;
a first iris positioned adjacent the housing;
a second iris positioned adjacent the housing; and
a spacer plate disposed between the first iris and the second iris;
wherein the spacer plate includes a first locking element disposed radially inward of an outer circumference of the spacer plate and a second locking element disposed radially inward of the outer circumference of the spacer plate;
wherein the first locking element is engaged with the first iris to selectively lock the first iris in a first open position and a first closed position;
wherein the second locking element is engaged with the second iris to selectively lock the second iris in a second open position and a second closed position.

14. The device of claim 13, wherein the first iris includes a first circumferential ring positioned coaxially relative to the central opening, a first plurality of arms extending radially inward from the first circumferential ring, and a first lever extending radially outward from the first circumferential ring; and
wherein the second iris includes a second circumferential ring positioned coaxially relative to the central opening, a second plurality of arms extending radially inward from the second circumferential ring, and a second lever extending radially outward from the second circumferential ring.

15. The device of claim 14, wherein the first locking element includes a first deflectable tab extending radially relative to the central opening and a first rounded projection extending from the first deflectable tab in a first direction parallel to a central longitudinal axis of the housing; and
wherein the second locking element includes a second deflectable tab extending radially relative to the central opening and a second rounded projection extending from the second deflectable tab in a second direction parallel to the central longitudinal axis of the housing.

16. The device of claim 15, wherein the second direction is opposite the first direction.

17. The device of claim 13, wherein more force is required to unlock the first iris from the first open position than to lock the first iris in the first open position, and more force is required to unlock the first iris from the first closed position than to lock the first iris in the first closed position.

18. The device of claim 13, wherein more force is required to unlock the second iris from the second open position than to lock the second iris in the second open position, and more force is required to unlock the second iris from the second closed position than to lock the second iris in the second closed position.

19. The device of claim 13, wherein the first locking element and the second locking element are monolithically formed with the spacer plate.

20. A device for radially compressing a stent, comprising:
a housing including a central opening;
a first iris positioned adjacent the housing, wherein the first iris includes a first circumferential ring positioned coaxially relative to the central opening, a first plurality of arms extending radially inward from the first circumferential ring, and a first lever extending radially outward from the first circumferential ring;
a second iris positioned adjacent the housing, wherein the second iris includes a second circumferential ring positioned coaxially relative to the central opening, a second plurality of arms extending radially inward from the second circumferential ring, and a second lever extending radially outward from the second circumferential ring; and
a spacer plate disposed between the first iris and the second iris;
wherein the spacer plate includes a first locking element disposed radially inward of an outer circumference of the spacer plate and a second locking element disposed radially inward of the outer circumference of the spacer plate, the second locking element being circumferentially spaced apart from the first locking element;
wherein the first locking element is engaged with the first circumferential ring to selectively lock the first iris in a first open position and a first closed position;
wherein the second locking element is engaged with the second circumferential ring to selectively lock the second iris in a second open position and a second closed position.
